(19) United States
(12) Patent Application Publication
(10) Pub. No.: US 2025/0312550 A1
(43) Pub. Date: Oct. 9, 2025

(54) PERIPHERAL DETECTION METHODS AND APPARATUS

(71) Applicant: ResMed Asia Pte. Ltd., Singapore (SG)

(72) Inventors: Lik Tze SEET, Singapore (SG); Garth BERRIMAN, Sydney (AU)

(21) Appl. No.: 18/866,925

(22) PCT Filed: May 18, 2023
(86) PCT No.: PCT/NZ2023/050054

§ 371 Date: Nov. 18, 2024

(30) Foreign Application Priority Data
May 19, 2022 (AU) 2022901340

(51) Int. Cl. A61M 16/08

(52) U.S. Cl. CPC A61M 16/0816; A61M 2205/14; A61M 2205/3317; A61M 2205/3327

(57) ABSTRACT

Disclosed are systems methods and devices for detecting the connection, disconnection, or connection quality of peripheral devices to a respiratory pressure therapy (RPT) device. Examples of the technology include the use of passive and magnetic components. On detection, the RPT device can perform one or more actions including the automated starting or stopping of the RPT device.

### 1 CROSS REFERENCE TO RELATED APPLICATIONS

This application claims the benefit of Australian Provisional Patent Application No. 2022901340, the contents of which are herein incorporated by reference in their entirety.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 2.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO2 from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 2.2.2.3 Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH2O relative to ambient pressure. For other forms of therapy, such as the delivery of oxygen, the patient interface may not include a seal sufficient to facilitate delivery to the airways of a supply of gas at a positive pressure of about 10 cmH2O. For flow therapies such as nasal HFT, the patient interface is configured to insufflate the nares but specifically to avoid a complete seal. One example of such a patient interface is a nasal cannula.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

Air pressure generators are known in a range of applications, e.g. industrial-scale ventilation systems. However, air pressure generators for medical applications have particular requirements not fulfilled by more generalised air pressure generators, such as the reliability, size and weight requirements of medical devices. In addition, even devices designed for medical treatment may suffer from shortcomings, pertaining to one or more of: comfort, noise, ease of use, efficacy, size, weight, manufacturability, cost, and reliability.

An example of the special requirements of certain RPT devices is acoustic noise.

Table of noise output levels of prior RPT devices (one specimen only, measured using test method specified in ISO 3744 in CPAP mode at 10 cmH2O).

| | A-weighted sound | Year |
|---|---|---|
| RPT Device name | pressure level dB(A) | (approx.) |
| C-Series TangoTM | 31.9 | 2007 |
| C-Series TangoTM with Humidifier | 33.1 | 2007 |
| S8 EscapeTM II | 30.5 | 2005 |
| S8 EscapeTM II with H4iTM | 31.1 | 2005 |
| Humidifier | | |
| S9 AutoSetTM | 26.5 | 2010 |
| S9 AutoSetTM with H5i Humidifier | 28.6 | 2010 |

One known RPT device used for treating sleep disordered breathing is the S9 Sleep Therapy System, manufactured by ResMed Limited. Another example of an RPT device is a ventilator. Ventilators such as the ResMed Stellar™ Series of Adult and Paediatric Ventilators may provide support for invasive and non-invasive non-dependent ventilation for a range of patients for treating a number of conditions such as but not limited to NMD, OHS and COPD.

The ResMed Elisée™ 150 ventilator and ResMed VS III™ ventilator may provide support for invasive and non-invasive dependent ventilation suitable for adult or paediatric patients for treating a number of conditions. These ventilators provide volumetric and barometric ventilation modes with a single or double limb circuit. RPT devices typically comprise a pressure generator, such as a motor-driven blower or a compressed gas reservoir, and are configured to supply a flow of air to the airway of a patient. In some cases, the flow of air may be supplied to the airway of the patient at positive pressure. The outlet of the RPT device is connected via an air circuit to a patient interface such as those described above.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

The design of an air circuit presents a number of challenges. The air circuit must allow the patient to move as freely as possible in order to avoid the feeling of being “tethered” to the bed. Air circuits with some ability to extend along their length may be useful for this reason. However, if the air circuit reacts to internal changes in pressure by deforming then this may be uncomfortable for the patient and may also affect the RPT's ability to accurately estimate one or more variables at the patient interface, for example the pressure at the interface and/or a flow rate of a leak.

The weight of the air circuit should be as low as practical to reduce the tendency to pull the patient interface away from the patient's face. However, the air circuit must have a sufficiently large internal diameter to have a sufficiently low impedance to flow at the required flow rates, and must also be sufficiently rigid to avoid collapsing under the weight of the patient.

One method of increasing the crush resistance of the air circuit is to provide a ribbed shape to the external surface of the air circuit, for example a helical rib. Such ribs are typically substantially circular in cross-section. However, this formation may be prone to catching and/or may create an unpleasant noise when dragged across a surface (for example if the tube is dragged across an item of bedroom furniture when the patient rolls over).

One solution which seeks to overcome the disadvantages of a standard air circuit is the use of a lighter, more flexible tube (sometimes called a “short tube”) between the patient interface and the main air circuit. In some examples the short tube may have a “concertina” cross-section to allow it to be easily extensible.

A short tube may be lighter, more easily crushed and/or more prone to deformation due to internal pressure than a main air circuit, but these disadvantages may be mitigated by the relatively short length of the short tube (typically around 50 cm) and the fact that it is located near the patient interface.

One disadvantage of systems which use a short tube is the need for a connector between the short tube and the main air circuit. The connector may be relatively heavy and may also add to the cost of the system.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 2.2.3.5 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 2.2.3.6 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

### 2.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiogramalectrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 2.2.5 Treatment Effectiveness

During treatment of respiratory illnesses, there is a need to maintain a therapeutic delivery of breathable gas to the patient interface. Effective delivery of these breathable gases requires each component of the respiratory therapy system to be working together correctly to provide effective treatment. However, in practice, various factors can reduce the effectiveness of the treatment being provided.

Many respiratory therapies are conducted over periods of several hours, for example CPAP therapy for treatment of sleep apnea is a therapy which is often performed over night to assist with keeping the patient's airways open and unobstructed during sleep.

There are number of external factors which can influence whether effective treatment is provided by a respiratory therapy system. For example, the air circuit may become obstructed or disconnected during use, such as due to a patient moving and obstructing the air conduit while asleep. Other examples include the patient interface shifting on the patient's face, and/or the seal with the patient's airways being compromised.

Some of these external factors can be detected and accounted for algorithmically. For example, a pressure drop in the respiratory therapy system may be at least partially counteracted by the RPT device increasing the pressure of the provided breathable gas. However, while pressure drops can be detected, it can be difficult to correlate the drop to any specific cause. Therefore, it can be difficult to determine whether effective treatment is still being provided by the system.

Some of the algorithms which attempt to account for changes to the respiratory system can result in negative outcomes for the patient. For example, in humidified systems attempting to correct for a drop in air pressure by increasing the flow rate from the flow generator can result in the water contained in the humidifier tank being depleted more rapidly. Once depleted, the patient must either refill the humidifier tank, which is inconvenient, or potentially suffer discomfort due to drying of their airways. Another negative outcome of increasing air pressure or flow is often an increase is the noise generated by the flow generator, and the noise generated by air escaping from components of the respiratory therapy system. This can result in an inability to sleep or reduced quality of sleep both for the patient, and any others sleeping nearby.

Some patients will also use several different patient interfaces with their respiratory therapy system. This can help to relieve or reduce the occurrence of pressure sores caused by the patient interface contacting areas of the patients face for extended periods of time. These interfaces can require different flow generator settings in order to deliver an optimised respiratory therapy treatment.

It is an object of the present invention to at least partially address one or more of the foregoing issues, or at least provide the public with a useful choice.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises an air circuit for a respiratory therapy system. The air circuit may comprise a conduit configured to receive a flow of breathable gas from a respiratory therapy device in use. The air circuit may further comprise a first cuff provided to a first end of the conduit to facilitate connection to a peripheral device in use. The air circuit may further comprise a second cuff provided to a second end of the conduit to facilitate connection to the respiratory therapy device in use. The air circuit may further comprise a detection circuit configured to facilitate the detection of one or more components connected to the air circuit. The detection circuit may be further configured to detect the presence absence of the peripheral device in use, and to communicate information regarding the presence or absence of the peripheral device to the respiratory therapy device.

Another aspect of one form of the present technology is a respiratory therapy system. The respiratory therapy system may comprise: a respiratory therapy device configured to generate a flow of breathable gas. The respiratory therapy system may further comprise an air circuit configured to receive the flow of breathable gas from the respiratory therapy device. The respiratory therapy system may further comprise a patient interface configured to facilitate delivery of the breathable gas to a patient's airways in use. The air circuit may further comprise a detection circuit which is configured to detect one or more features of the patient interface, and communicate information regarding the patient interface to the respiratory therapy device. The respiratory therapy device may further be configured to provide the flow of breathable gas when the information indicates that the patient interface is connected to the air circuit. The respiratory therapy device may further be configured to not provide the flow of breathable gas when the information indicates that the patient interface is disconnected from the air circuit.

Another aspect of one form of the present technology is a patient interface for use in a respiratory therapy system. The patient interface may comprise: a plenum chamber enclosing a volume of space. The patient interface may further comprise a connection port, configured to receive the flow of breathable gas from an air circuit in use and to communicate the flow of breathable gas to the plenum chamber. The patient interface may further comprise a seal-forming structure configured to provide an air seal around an entrance to a patient's airways and to direct the flow of breathable gas from the plenum chamber to the patient's airways in use. The patient interface may further comprise at least one identifying feature which allows a detection circuit within the air circuit to detect when the patient interface is connected to the air circuit.

Another aspect of one form of the present technology is a respiratory therapy device. The respiratory therapy device may comprise: a flow generator configured to generate a flow of breathable gas. The respiratory therapy device may further comprise an outlet configured to communicate the flow of breathable gas to an air circuit in use. The flow generator may further comprise a detection circuit configured to detect the connection or disconnection of the air circuit from the outlet. The respiratory therapy system may further be configured to provide the flow of breathable gas when the air circuit is connected, and not provide the flow of breathable gas when the air circuit is disconnected.

In examples of the technology, the detection circuit may comprise a switch. For example, the switch may be configured to engage with the identifying feature to close or open an electrical circuit.

In examples of the technology, the detection circuit may comprise two or more electrical contacts which in use are electrically connected or disconnected by the identifying feature on the patient interface when the patient interface is connected to the air circuit. For example, the feature may include an electrical short, one or more passive electronic components such as resistors, capacitors and inductors, active electronic components including rectifiers, regulators, microcontrollers, resonant circuits, signal generators, or switching elements such as transistors.

In other examples, the detection circuit may comprise one or more components of a Radio Frequency Identification (RFID) circuit. For example, the detection circuit may comprise a complete RFID reading circuit, or alternatively the antenna of an RFID reading circuit. The RFID reading circuit may be configured to communicate with a RFID feature such as a transceiver or tag in the patient interface to facilitate detection of the patient interface.

In other examples, the detection circuit may comprise one or more components configured to detect the presence of a magnetic field. For example, the detection circuit may comprise a Hall-effect sensor or reed switch. Accordingly, the identifying feature may be a device configured to provide a magnetic field such as a permanent magnet or electromagnet.

An aspect of certain forms of the present technology is a medical device that is easy to use, e.g. by a person who does not have medical training, by a person who has limited dexterity, vision or by a person with limited experience in using this type of medical device.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

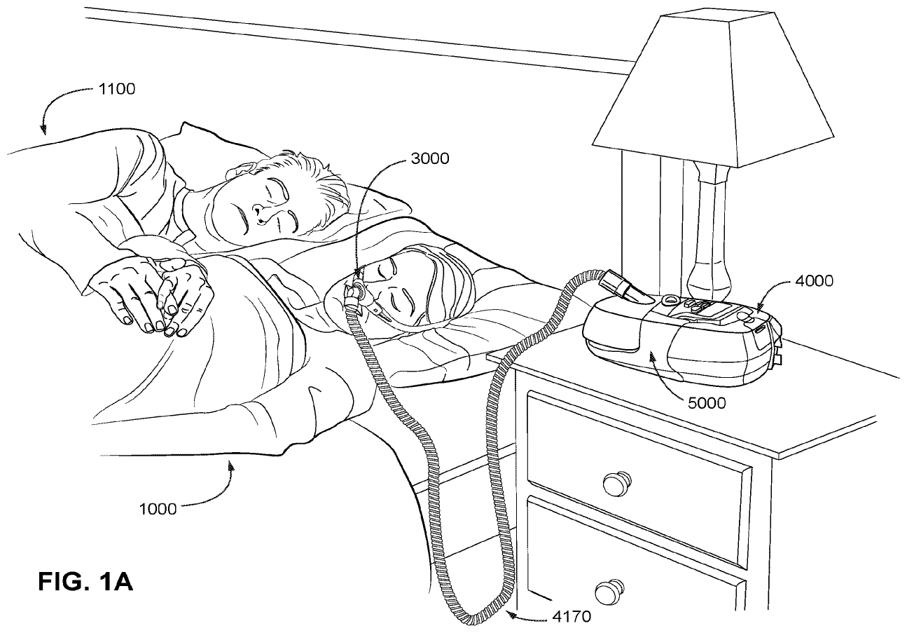

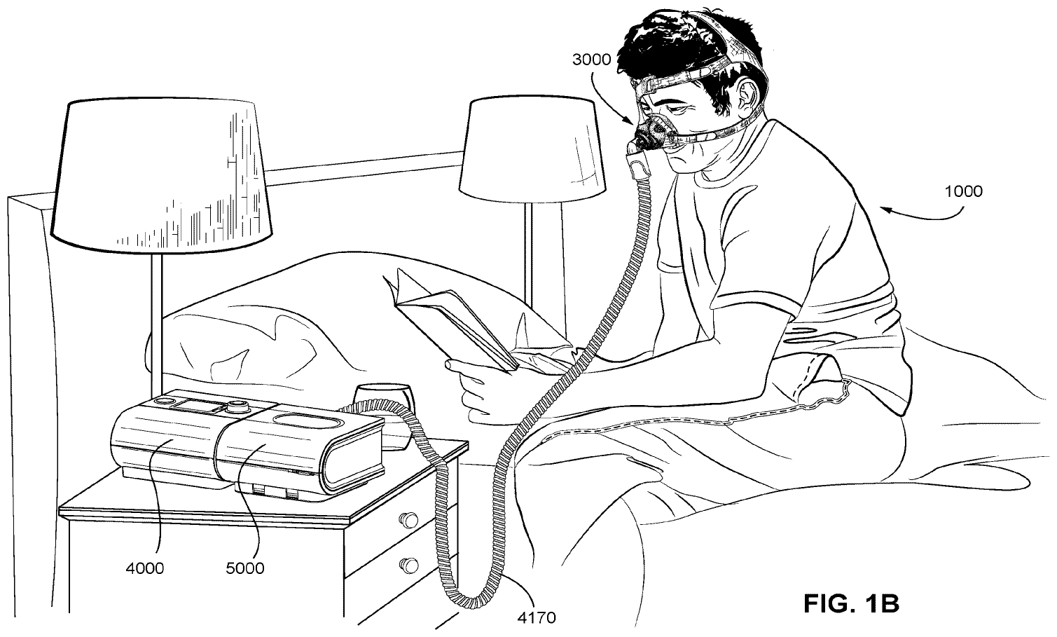

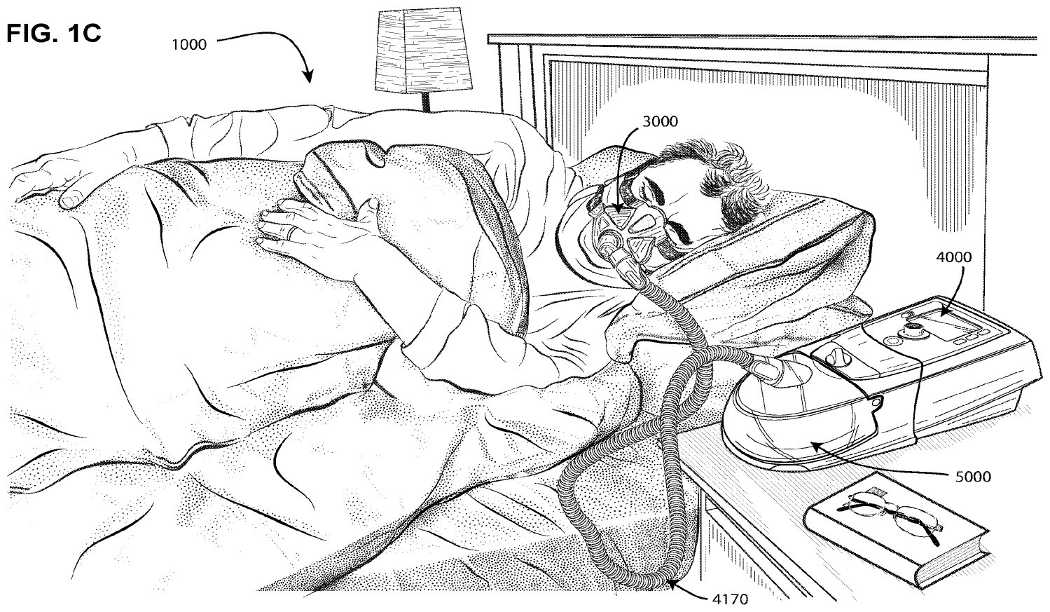

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

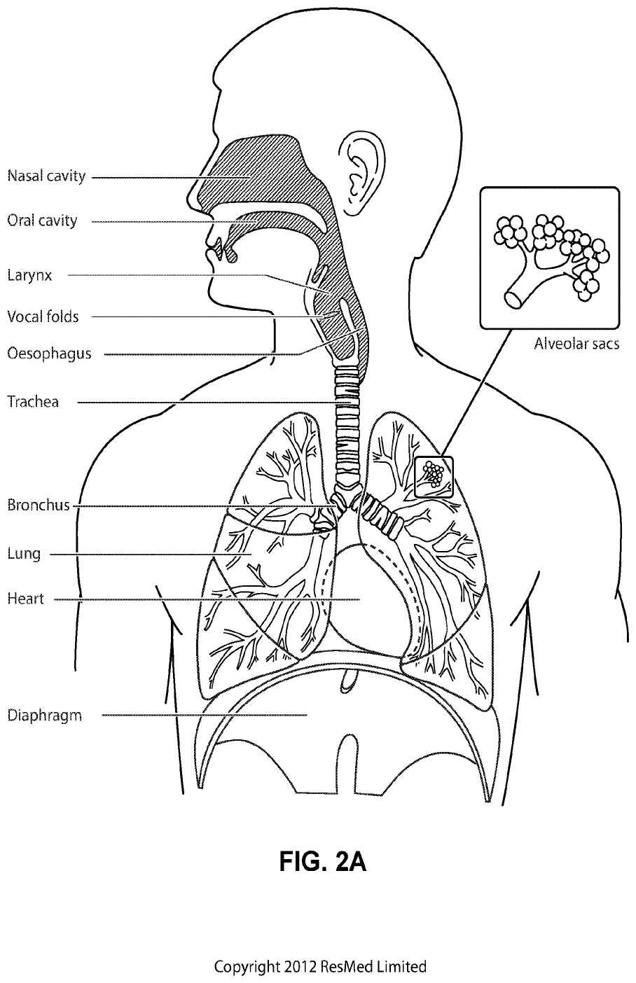

FIG. 2A shows an overview of a human respiratory system including the nasal and oral cavities, the larynx, vocal folds, oesophagus, trachea, bronchus, lung, alveolar sacs, heart and diaphragm.

### 4.3 Patient Interface

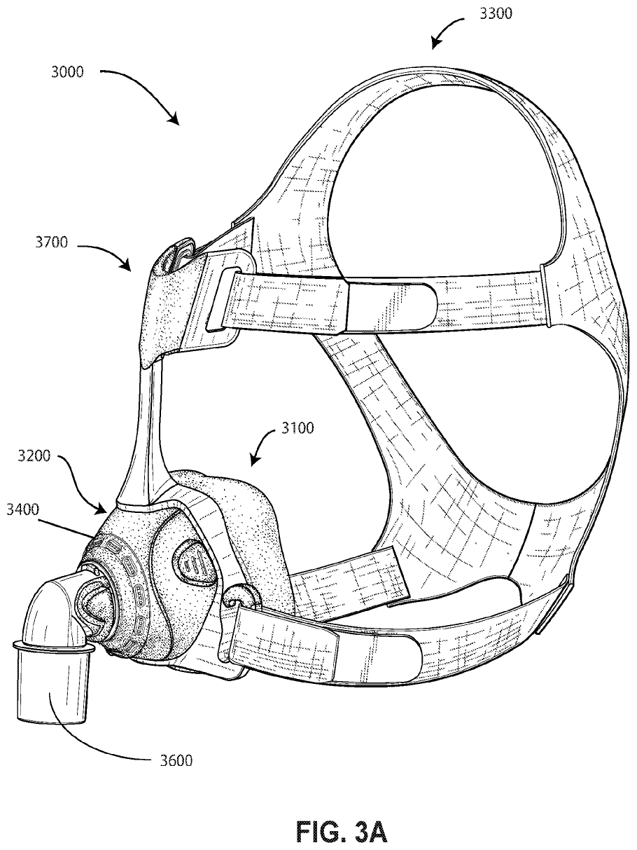

FIG. 3A shows a patient interface in the form of a nasal mask in accordance with one form of the present technology.

### 4.4 RPT Device

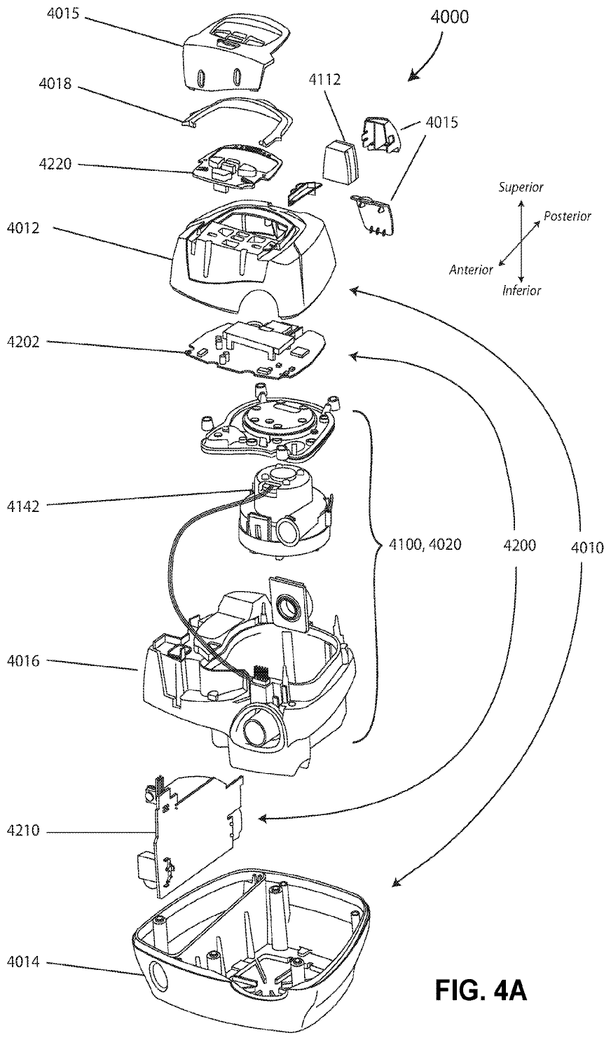

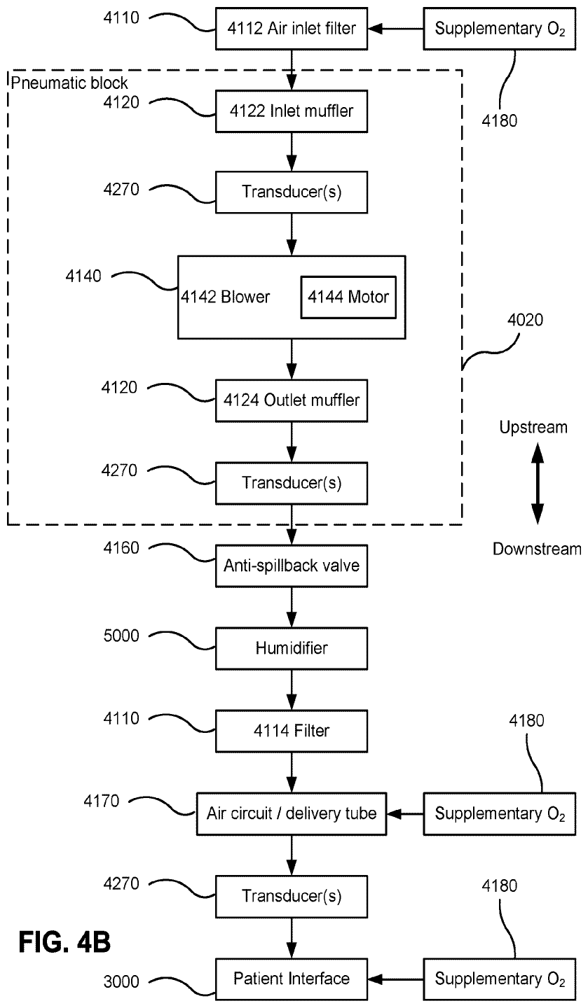

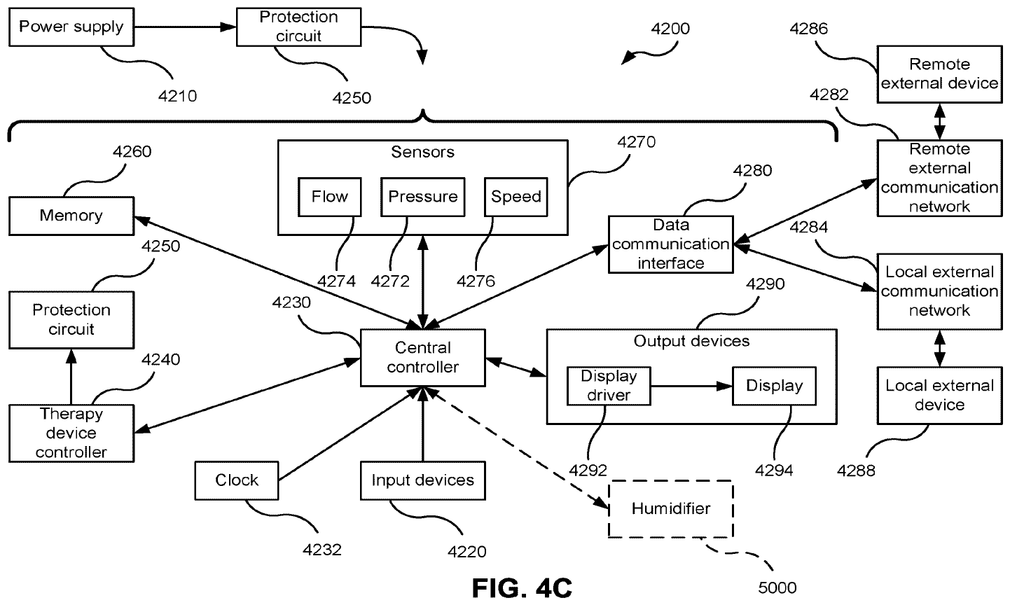

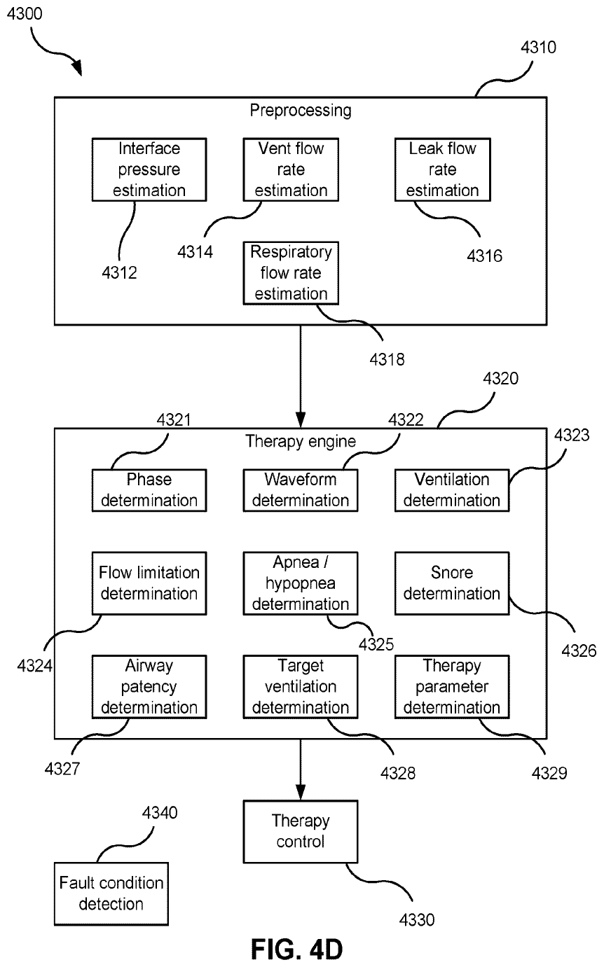

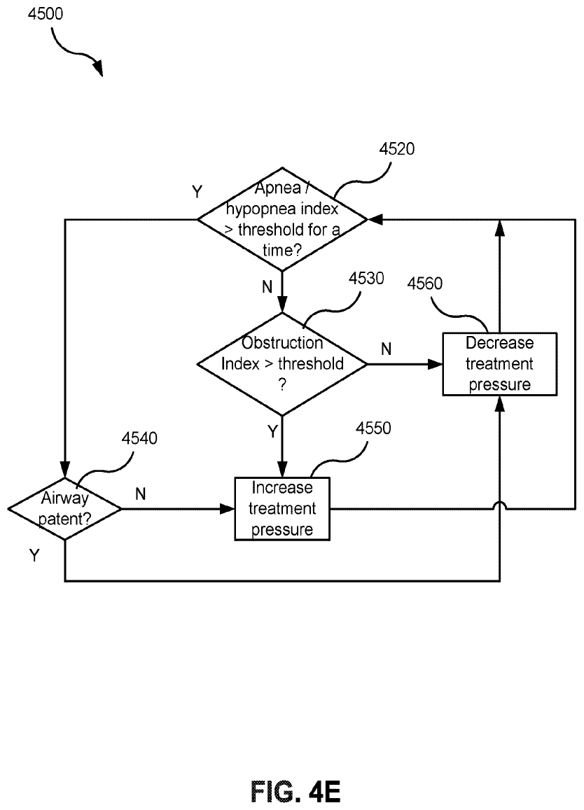

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

FIG. 4C is a schematic diagram of the electrical components of an RPT device in accordance with one form of the present technology.

FIG. 4D is a schematic diagram of the algorithms implemented in an RPT device in accordance with one form of the present technology.

FIG. 4E is a flow chart illustrating a method carried out by the therapy engine module of FIG. 4D in accordance with one form of the present technology.

### 4.5 Humidifier

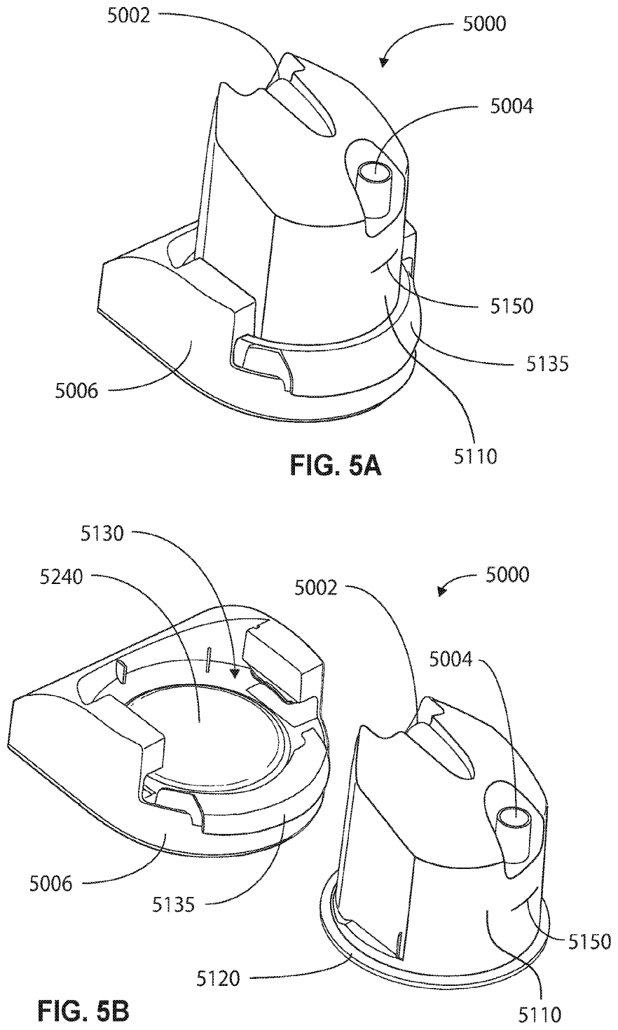

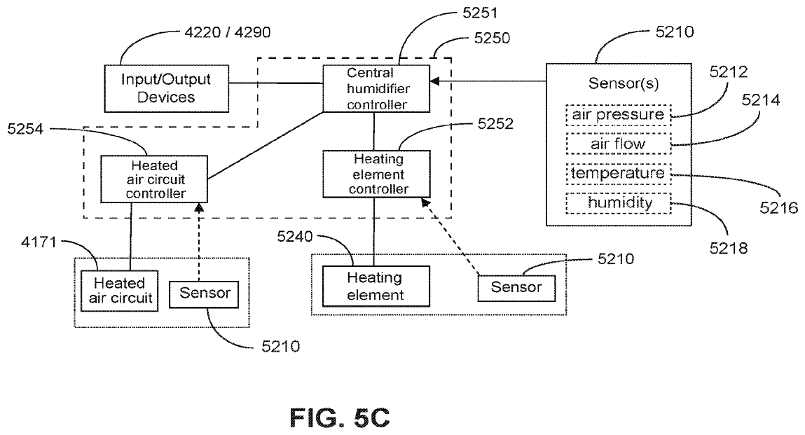

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

FIG. 5C shows a schematic of a humidifier in accordance with one form of the present technology.

### 4.6 Breathing Waveforms

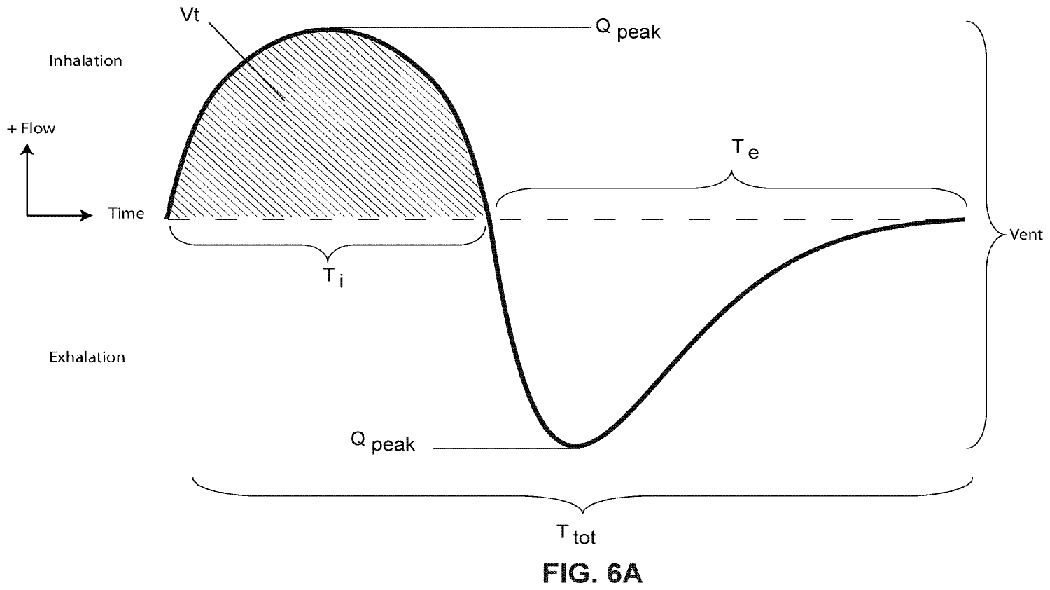

FIG. 6A shows a model typical breath waveform of a person while sleeping.

### 4.7 Peripheral Detection

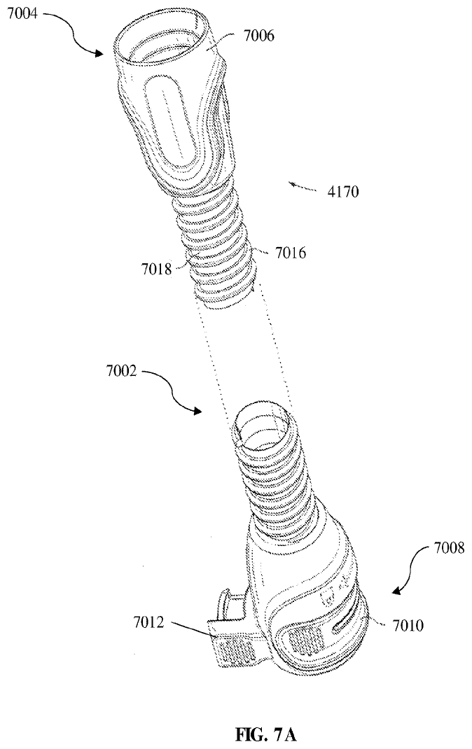

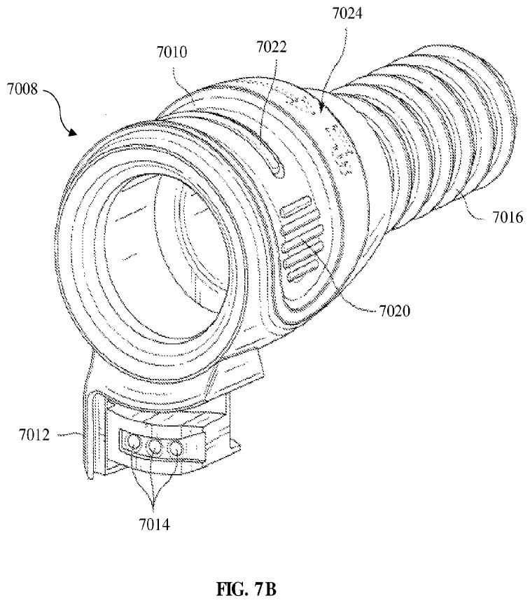

FIG. 7A shows an example of an air circuit in accordance with one form of the present technology.

FIG. 7B shows a perspective view of an end of the air circuit of FIG. 7A.

FIG. 7C shows a side view of the end of the air circuit of FIG. 7A.

FIG. 7D shows a perspective view of an end of an air circuit of FIG. 7A.

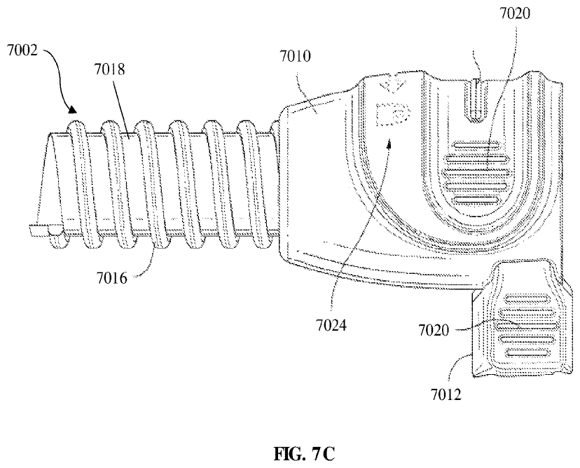

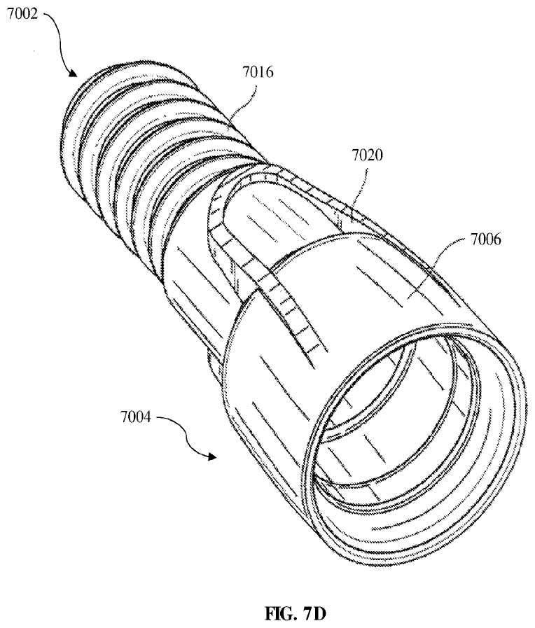

FIG. 8 shows an RPT device in accordance with the present technology.

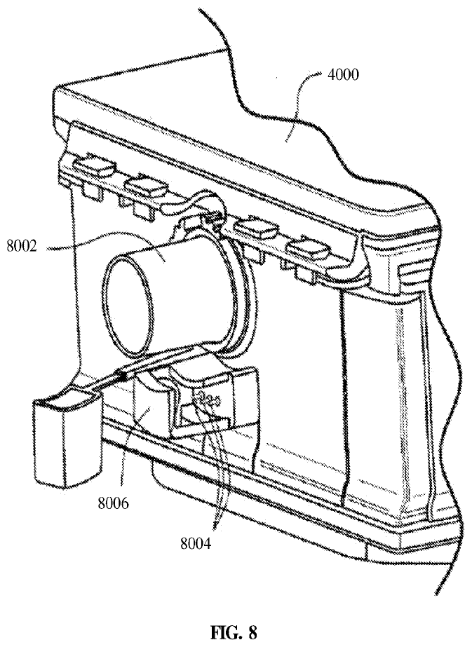

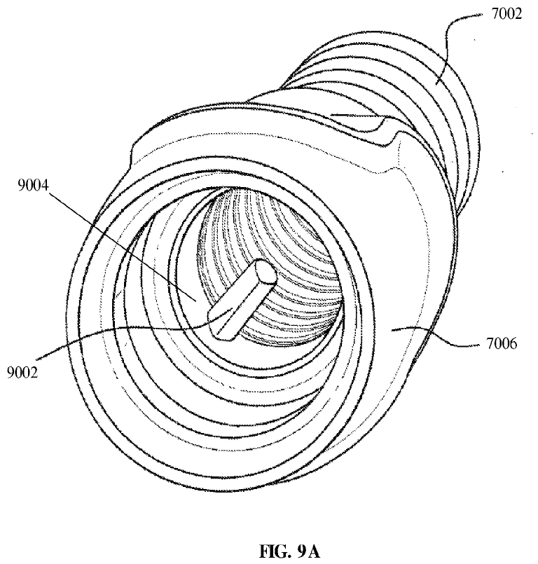

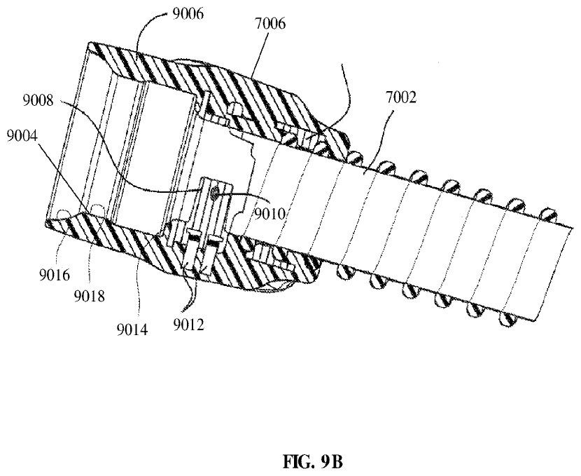

FIG. 9A shows an end of an air circuit comprising a detection circuit in accordance with the present technology.

FIG. 9B shows a cross sectional view of the end air circuit shown in FIG. 9A.

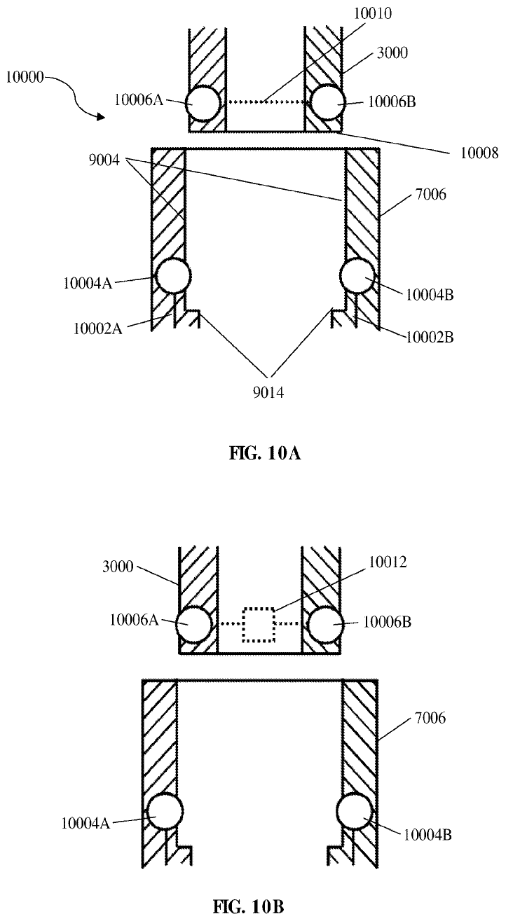

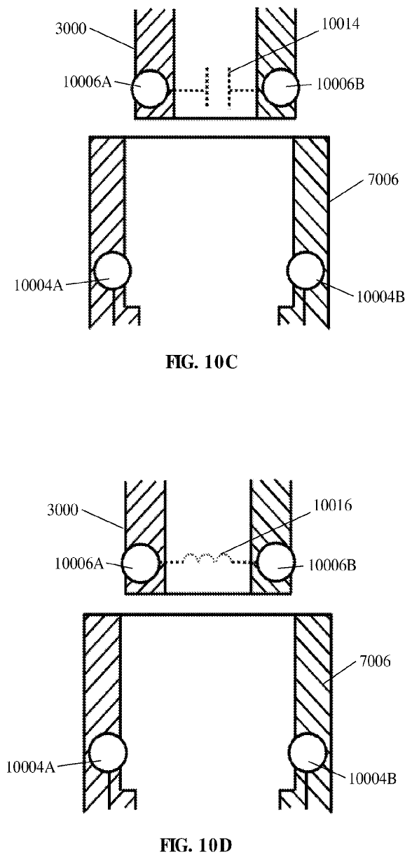

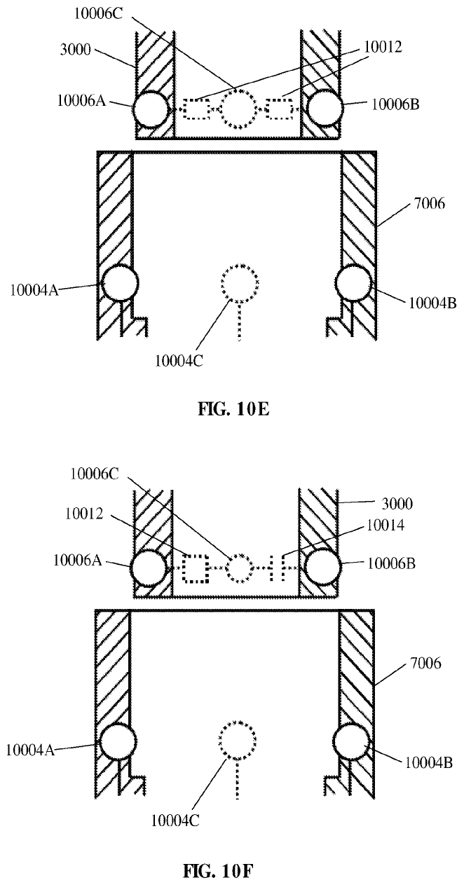

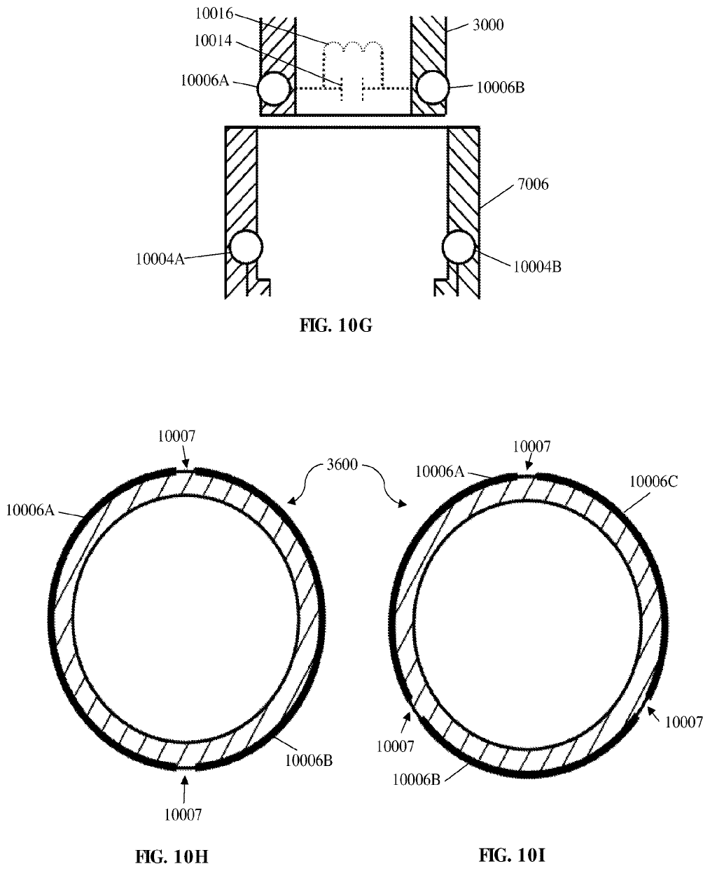

FIG. 10A shows a cross sectional view of an exemplary patient interface to air circuit connection using a conductor to facilitate detection.

FIG. 10B shows a cross sectional view of an exemplary patient interface to air circuit connection using a resistor to facilitate detection.

FIG. 10C shows a cross sectional view of an exemplary patient interface to air circuit connection using a capacitor to facilitate detection.

FIG. 10D shows a cross sectional view of an exemplary patient interface to air circuit connection using a inductor to facilitate detection.

FIG. 10E shows a cross sectional view of an exemplary patient interface to air circuit connection using a plurality of resistors to facilitate detection.

FIG. 10F shows a cross sectional view of an exemplary patient interface to air circuit connection using resistors and capacitors to facilitate detection.

FIG. 10G shows a cross sectional view of an exemplary patient interface to air circuit connection using capacitors and inductors to facilitate detection.

FIG. 10H shows an end view of a connection port illustrating an example of how terminals may be provided to a patient interface in accordance with the present technology.

FIG. 10I shows an end view of a connection port illustrating a further example of how terminals may be provided to a patient interface in accordance with the present technology.

FIG. 10J shows a cross sectional view of an exemplary patient interface to air circuit connection using a plurality of terminals to determine insertion depth in accordance with the present technology.

FIG. 10K shows a cross sectional view of an exemplary patient interface to air circuit connection using a longitudinally positioned terminals to determine insertion depth in accordance with the present technology.

FIG. 11A shows a cross sectional view of an exemplary patient interface to air circuit connection using a magnetic detection technologies in accordance with the present technology.

FIG. 11B shows a cross sectional view of an exemplary patient interface to air circuit connection using a magnetic detection technologies in accordance with the present technology.

FIG. 11C shows a cross sectional view of an exemplary patient interface to air circuit connection using magnetic detection technologies in accordance with the present technology.

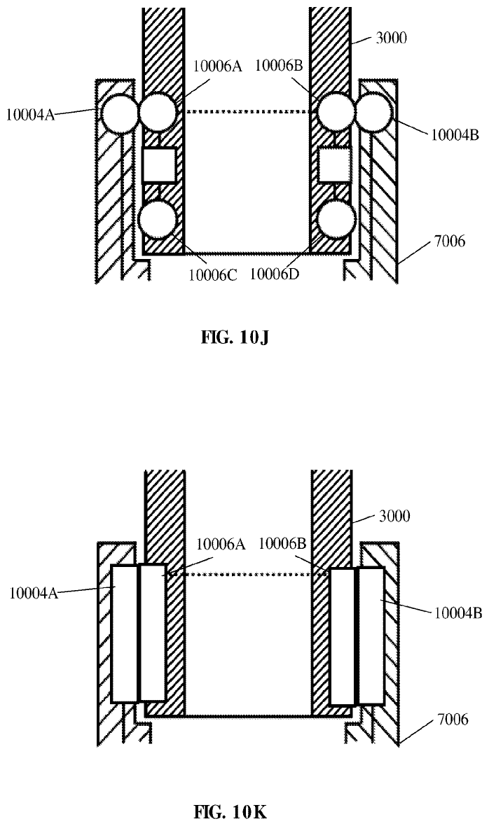

FIG. 12 shows a cross sectional view of an exemplary patient interface to air circuit connection using radio frequency (RF) detection technologies in accordance with the present technology.

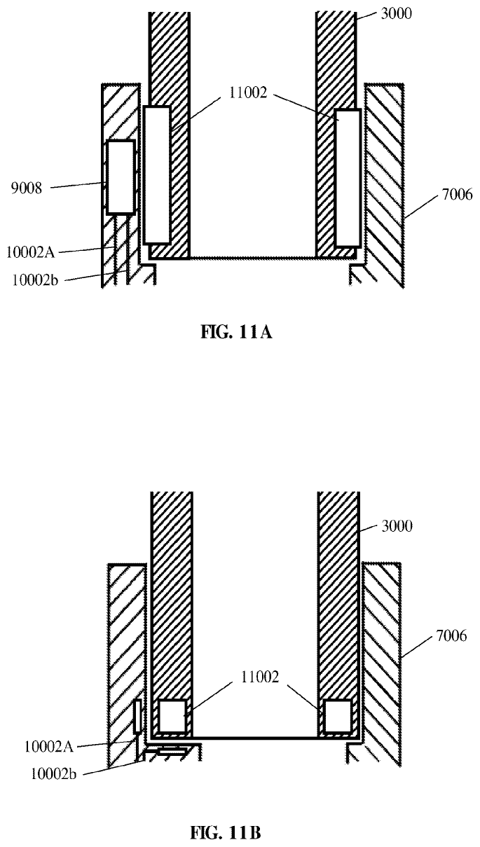

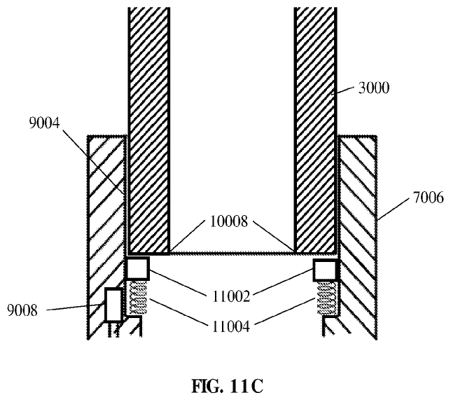

FIG. 13 shows a cross sectional view of an exemplary patient interface to air circuit connection using mechanical detection technologies in accordance with the present technology.

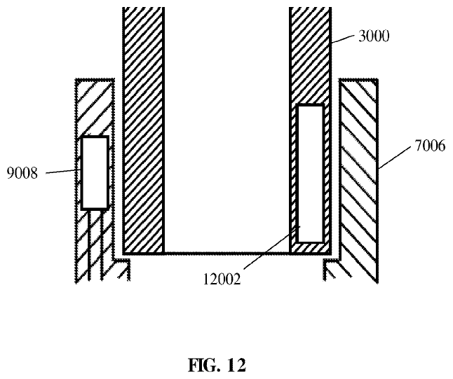

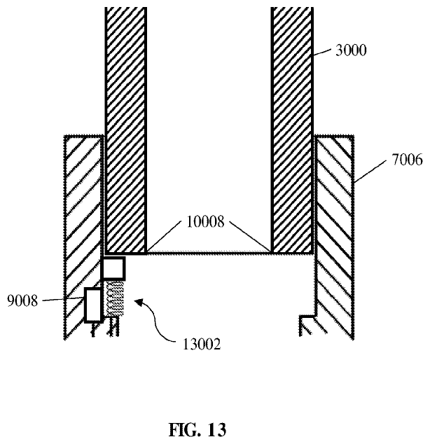

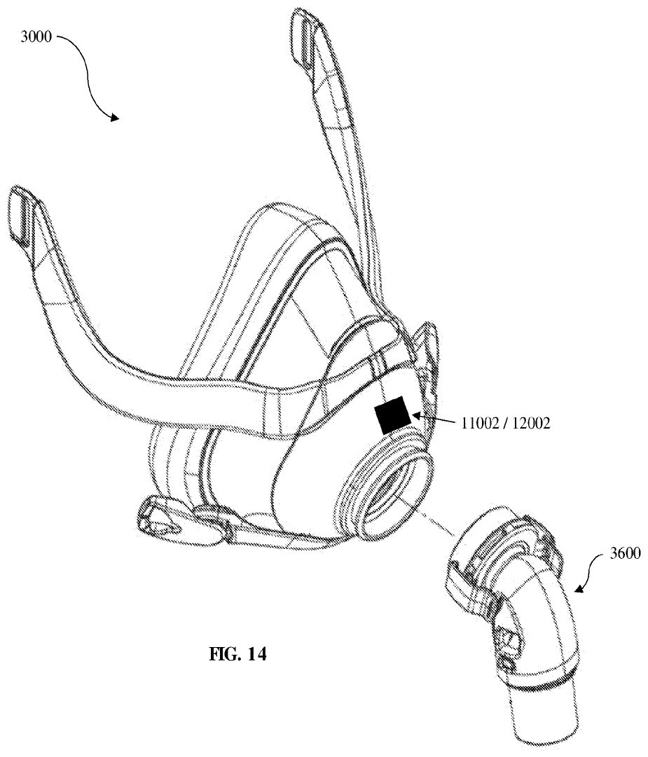

FIG. 14 shows a perspective view of an exemplary patient interface in accordance with the present technology.

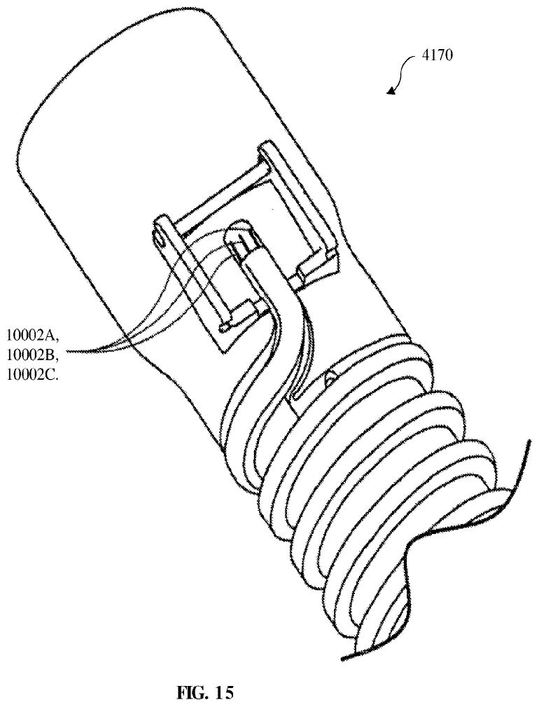

FIG. 15 shows a perspective view of a heated air circuit comprising a plurality of wires in accordance with the present technology.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 5.3 Patient Interface

A non-invasive patient interface 3000 in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

An unsealed patient interface 3800, in the form of a nasal cannula, includes nasal prongs 3810a, 3810b which can deliver air to respective nares of the patient 1000 via respective orifices in their tips. Such nasal prongs do not generally form a seal with the inner or outer skin surface of the nares. This type of interface results in one or more gaps that are present in use by design (intentional) but they are typically not fixed in size such that they may vary unpredictably by movement during use. This can present a complex pneumatic variable for a respiratory therapy system when pneumatic control and/or assessment is implemented, unlike other types of mask-based respiratory therapy systems. The air to the nasal prongs may be delivered by one or more air supply lumens 3820a, 3820b that are coupled with the nasal cannula-type unsealed patient interface 3800. The lumens 3820a, 3820b lead from the nasal cannula-type unsealed patient interface 3800 to a respiratory therapy device via an air circuit. The unsealed patient interface 3800 is particularly suitable for delivery of flow therapies, in which the RPT device generates the flow of air at controlled flow rates rather than controlled pressures. The “vent” or gap at the unsealed patient interface 3800, through which excess airflow escapes to ambient, is the passage between the end of the prongs 3810a and 3810b of the nasal cannula-type unsealed patient interface 3800 via the patient's nares to atmosphere.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure with respect to ambient, for example a positive pressure of at least 2, 4, 6, 10 or 20 cmH2O with respect to ambient.

### 5.3.1 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO₂ by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel. Examples of swivel elbow assemblies may be substantially as described in International Publication No. WO 2017/049357 A1, the entire contents of which are incorporated herein by reference.

### 5.3.2 Decoupling Structure(s)

In one form the patient interface 3000 includes at least one decoupling structure, for example, a swivel or a ball and socket. The decoupling structure may take the form of an elbow, swivel, ball and socket, or other bent (e.g. curved) component. The elbow may be configured to rotate relative to the patient interface, thereby at least partially decoupling the rotational forces applies to the air circuit 4170 from the patient interface 3000.

### 5.3.3 Connection Port

Connection port 3600 allows for connection to the air circuit 4170.

In use the air circuit 4170 may connect to the connection port 3600 to allow for delivery of the flow of breathable gas from the RPT device to the patient's airways. The connection port may be provided as part of a decoupling structure.

### 5.3.4 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

### 5.3.4.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 5.3.4.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 5.3.4.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 5.3.4.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 5.3.4.5 Forehead Region

In one form, the seal-forming structure that forms a seal in use on a forehead region of the patient's face. In such a form, the plenum chamber may cover the eyes in use.

### 5.3.4.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 5.3.5 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

### 5.3.6 Positioning and Stabilising Structure

The seal-forming structure 3100 of the patient interface 3000 of the present technology may be held in sealing position in use by the positioning and stabilising structure 3300.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilizing structure 3300, each being configured to provide a retaining force to correspond to a different size and/or shape range. For example the system may comprise one form of positioning and stabilizing structure 3300 suitable for a large sized head, but not a small sized head, and another. suitable for a small sized head, but not a large sized head.

### 5.3.7 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 5.4 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 6 cmH2O, or at least 10 cmH2O, or at least 20 cmH2O.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying air at positive pressure (e.g., a blower 4142), an outlet muffler 4124 and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 5.4.1 RPT Device Mechanical & Pneumatic Components

An RPT device may comprise one or more of the following components in an integral unit. In an alternative form, one or more of the following components may be located as respective separate units.

### 5.4.1.1 Air Filter(s)

An RPT device in accordance with one form of the present technology may include an air filter 4110, or a plurality of air filters 4110.

In one form, an inlet air filter 4112 is located at the beginning of the pneumatic path upstream of a pressure generator 4140.

In one form, an outlet air filter 4114, for example an antibacterial filter, is located between an outlet of the pneumatic block 4020 and a patient interface 3000 or 3800.

### 5.4.1.2 Muffler(s)

An RPT device in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface 3000 or 3800.

### 5.4.1.3 Pressure Generator

In one form of the present technology, a pressure generator 4140 for producing a flow, or a supply, of air at positive pressure is a controllable blower 4142. For example, the blower 4142 may include a brushless DC motor 4144 with one or more impellers. The impellers may be located in a volute. The blower may be capable of delivering a supply of air, for example at a rate of up to about 120 litres/minute, at a positive pressure in a range from about 4 cmH2O to about 20 cmH2O, or in other forms up to about 30 cmH2O when delivering respiratory pressure therapy. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 4140 may be under the control of the therapy device controller 4240.

In other forms, a pressure generator 4140 may be a piston-driven pump, a pressure regulator connected to a high pressure source (e.g. compressed air reservoir), or a bellows.

### 5.4.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 may be constructed and arranged to generate signals representing properties of the flow of air such as a flow rate, a pressure or a temperature at that point in the pneumatic path.

In one form of the present technology, one or more transducers 4270 may be located proximate to the patient interface 3000 or 3800.

In one form, a signal from a transducer 4270 may be filtered, such as by low-pass, high-pass or band-pass filtering.

### 5.4.1.4.1 Flow Rate Sensor

A flow rate sensor 4274 in accordance with the present technology may be based on a differential pressure transducer, for example, an SDP600 Series differential pressure transducer from SENSIRION.

In one form, a signal generated by the flow rate sensor 4274 and representing a flow rate is received by the central controller 4230.

### 5.4.1.4.2 Pressure Sensor

A pressure sensor 4272 in accordance with the present technology is located in fluid communication with the pneumatic path. An example of a suitable pressure sensor is a transducer from the HONEYWELL ASDX series. An alternative suitable pressure sensor is a transducer from the NPA Series from GENERAL ELECTRIC.

In one form, a signal generated by the pressure sensor 4272 and representing a pressure is received by the central controller 4230.

### 5.4.1.4.3 Motor Speed Transducer

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 5.4.1.5 Anti-Spill Back Valve

In one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

5.4.2 RPT device Electrical Components

### 5.4.2.1 Power Supply

A power supply 4210 may be located internal or external of the external housing 4010 of the RPT device 4000.

In one form of the present technology, power supply 4210 provides electrical power to the RPT device 4000 only. In another form of the present technology, power supply 4210 provides electrical power to both RPT device 4000 and humidifier 5000.

### 5.4.2.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form, the input device 4220 may be constructed and arranged to allow a person to select a value and/or a menu option.

### 5.4.2.3 Central Controller

In one form of the present technology, the central controller 4230 is one or a plurality of processors suitable to control an RPT device 4000.

Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 4230 is a dedicated electronic circuit.

In one form, the central controller 4230 is an application-specific integrated circuit. In another form, the central controller 4230 comprises discrete electronic components.

The central controller 4230 may be configured to receive input signal(s) from one or more transducers 4270, one or more input devices 4220, and the humidifier 5000.

The central controller 4230 may be configured to provide output signal(s) to one or more of an output device 4290, a therapy device controller 4240, a data communication interface 4280, and the humidifier 5000.

In some forms of the present technology, the central controller 4230 is configured to implement the one or more methodologies described herein, such as the one or more algorithms 4300 which may be implemented with processor-control instructions, expressed as computer programs stored in a non-transitory computer readable storage medium, such as memory 4260. In some forms of the present technology, the central controller 4230 may be integrated with an RPT device 4000. However, in some forms of the present technology, some methodologies may be performed by a remotely located device. For example, the remotely located device may determine control settings for a ventilator or detect respiratory related events by analysis of stored data such as from any of the sensors described herein.

### 5.4.2.4 Clock

The RPT device 4000 may include a clock 4232 that is connected to the central controller 4230.

### 5.4.2.5 Therapy Device Controller

In one form of the present technology, therapy device controller 4240 is a therapy control module 4330 that forms part of the algorithms 4300 executed by the central controller 4230.

In one form of the present technology, therapy device controller 4240 is a dedicated motor control integrated circuit. For example, in one form a MC33035 brushless DC motor controller, manufactured by ONSEMI is used.

### 5.4.2.6 Memory

In accordance with one form of the present technology the RPT device 4000 includes memory 4260, e.g., non-volatile memory. In some forms, memory 4260 may include battery powered static RAM. In some forms, memory 4260 may include volatile RAM.

Memory 4260 may be located on the PCBA 4202. Memory 4260 may be in the form of EEPROM, or NAND flash.

Additionally, or alternatively, RPT device 4000 includes a removable form of memory 4260, for example a memory card made in accordance with the Secure Digital (SD) standard.

In one form of the present technology, the memory 4260 acts as a non-transitory computer readable storage medium on which is stored computer program instructions expressing the one or more methodologies described herein, such as the one or more algorithms 4300.

### 5.4.2.7 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 5.4.2.7.1 Display Driver

A display driver 4292 receives as an input the characters, symbols, or images intended for display on the display 4294, and converts them to commands that cause the display 4294 to display those characters, symbols, or images.

### 5.4.2.7.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

### 5.4.3 RPT Device Algorithms

As mentioned above, in some forms of the present technology, the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and/or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs, such as with processor control instructions to be executed by one or more processor(s), stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 5.5 Air Circuit

An air circuit 4170 in accordance with an aspect of the present technology is a conduit or a tube constructed and arranged to allow, in use, a flow of air to travel between two components such as RPT device 4000 and the patient interface 3000 or 3800.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller 4230. One example of an air circuit 4170 comprising a heated wire circuit is described in U.S. Pat. No. 8,733,349, which is incorporated herewithin in its entirety by reference.

The air circuit 4170 may include one or more cuffs 7006, 7010 to facilitate connection of the air circuit 4170 to a patient interface 3000 and/or RPT device 4000.

### 5.6 Humidifier

### 5.6.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 5.7 Breathing Waveforms

FIG. 6A shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6 s, peak inspiratory flow rate Qpeak 0.4 L/s, exhalation time Te 2.4 s, peak expiratory flow rate Qpeak-0.5 L/s. The total duration of the breath, Ttot, is about 4 s. The person typically breathes at a rate of about 15 breaths per minute (BPM), with Ventilation Vent about 7.5 L/min. A typical duty cycle, the ratio of Ti to Ttot, is about 40%.

### 5.8 Respiratory Therapy Modes

Various respiratory therapy modes may be implemented by the disclosed respiratory therapy system, including CPAP, Bi-level therapy, and High flow therapy.

### 5.9 Peripheral Detection

Certain forms of the technology relate to the automated detection of peripherals connected to a flow generator. For example, these peripherals can include air circuits 4170, patient interfaces 3000, humidifiers 5000, and components thereof.

FIGS. 7A, 7B, 7C, 7D, 7E and 7F show an air circuit 4170 in accordance with the present technology. The air circuit includes a conduit 7002 for communicating a flow of breathable gas between a RPT device 4000, such as a flow generator, and a peripheral device, such as a patient interface 3000.

A first end 7004 of the conduit 7002 may be provided with a first cuff 7006 configured to physically and fluidly connect the conduit to other components of a respiratory therapy system, such as peripheral devices such as a patient interface 3000, or components thereof in use. For example, the first cuff 7006 may be configured to fluidly and/or mechanically connect to a connection port 3600, which may be provided on an elbow or swivel of a patient interface 3000.

In a number of applications of the technology, it may be advantageous for the first cuff 7006 to connect to a peripheral device such as a patient interface 3000 via a friction fit connection. In other words, the first cuff 7006 may releasably connect to the connection port of the patient interface 3000 via a press-fit connection, or a connection without a latching or locking mechanism. This may allow the air circuit to disconnect in the event that a pulling force is applied to the air circuit, such as the patient attempting to walk away from the flow generator while the air circuit and patient interface 3000 remain connected. Other examples where it may be advantageous to automatically disconnect the air circuit from the patient interface 3000 include if the flow generator were to fall, such as being knocked off a table, or when the patient needs to get up in the middle of the night.

In other forms, for example paediatric masks, it may be advantageous for the first end 7004 of the air circuit to connect to the peripheral device such as a patient interface 3000 using a locking mechanism such as clips, a snap-fit connection or a bayonet-type fitting.

A second end 7008 of the conduit 7002 may be configured to connect to an RPT device 4000 such as a flow generator in use, and may be provided with a second cuff 7010. In the examples shown the second cuff 7010 is provided with a first locking half 7012 which is configured to engage with a second locking half 8006 on the RPT device 4000 as shown in FIG. 8. The first locking half 7012 and second locking half 8006 together provide a locking mechanism which secures the second end 7008 of the air circuit 4170 to the RPT device 4000 in use. In the example shown the locking mechanism is configured such that rotation of the second cuff 7010 relative to the RPT device 4000 causes the first locking half 7012 to engage with or alternatively disengage from the second locking half 8006. Accordingly, when the locking mechanism is engaged, the air circuit is locked to or otherwise secured to the RPT device 4000 such that disconnection, whether intentionally or accidentally, is not possible without first rotating the second cuff 7010 in the opposite direction.

In the example shown clockwise rotation of the second cuff 7010 relative to the RPT device 4000 causes the locking mechanism to engage securing the air circuit 4170 to the RPT device. Counter-clockwise rotation of the second cuff 7010 relative to the RPT device 4000 causes the locking mechanism to disengage or otherwise release from the RPT device 4000 which allows removal of the air circuit 4170 from the RPT device 4000. However, in other forms, other locking mechanisms such as clips, and snap-fit connections, such as known to those skilled in the art, may be used to connect the second end 7008 of the conduit 7002 to the RPT device 4000. Furthermore, in some forms of the technology, the second cuff 7010 may be configured to not include a locking mechanism, and instead may use a friction fit, in a similar manner to the first cuff 7006.

In some examples of the technology the air circuit 4170 may also comprise terminals 7014. These terminals 7014 may correspond to electrical contacts 8004 on the RPT device 4000, as shown in FIG. 8, for example the terminals 7014 may be positioned such that the relative positioning of the electrical contacts 8004 on the RPT device 4000 to the positioning of the terminals on the air circuit 4170 may ensure that, when the air circuit 4170 is engaged with the RPT device, the terminals are in electrical communication with the electrical contacts 8004. For example, the electrical contacts 8004 on the RPT device 4000 can be configured to engage with the terminals on the air circuit 4170 when the second cuff 7008 is connected to the RPT device 4000, and/or when the first locking half 7012 is engaged with the second locking half 8006.

In use the electrical connection of the electrical contacts 8004 to the terminals 7014 may facilitate delivery of current from the RPT device 4000 to a heated wire circuit within the air circuit 4170, thereby providing a heated air circuit 4170, as should be familiar to those skilled in the art. One example of a heated air circuit 4170 is shown in FIG. 15. The heated wire circuit 4170 may include conductors 10002A, 10002B, 10002C positioned along the length of the conduit, for example the conductors may be positioned within the ribs 7016 of the helical conduit 7002, or in the valleys 7018 between the ribs 7016.

A further use of the electrical terminals in some forms of air circuit 4170 may be to facilitate communication with a thermistor, or other suitable temperature measuring circuits within the air circuit 4170. The thermistor may allow for the temperature within the air circuit 4170 to be measured, for example at the first end 7004 of the air circuit 4170, adjacent to the peripheral device, such that temperature control adjustments may be made within the RPT device 4000 or humidifier to maintain an optimal temperature at the peripheral device. Further details of the use of thermistors within air circuits 4170 can be found in United States Patent Publication No. 2010/0116272A1 which is herein incorporated by reference in its entirety. In an example described in this document, communication with the thermistor may be achieved using dedicated wires within the air circuit 4170 that are separate to the heated wire circuit.

It should be appreciated that the air circuits described herein can further include features such as grip portions 7020, identifying components 7022 such as colour coding or labels, and markings 7024, such as connection instructions.

FIG. 8 shows an example RPT device 4000 which acts as a flow generator configured to provide a flow of breathable gas in use. The RPT device 4000 comprises a tubular connector 8002 which serves as the outlet of the flow of breathable gas from the RPT device 4000. In use, the second cuff 7010 of the air circuit 4170 is configured to engage with the tubular connector 8002 in order to facilitate the delivery of the flow of breathable gas through the air circuit 4170 to the peripheral device or patient interface 3000.

The RPT device 4000 of FIG. 8 also comprises electrical contacts 8004 in the form of pins which engage with the electrical terminals on the air circuit 4170 to enable the transfer of power and/or communication between the air circuit 4170 and RPT device in use, for example, by using the heated wire circuit or other electrical conductors within the air circuit 4170. An example of an air circuit 4170 comprising a heated wire circuit is shown in FIG. 15.

The RPT device 4000 of FIG. 8 includes a second locking half 8006, which is configured to engage with a first locking half 7012 on the air circuit 4170 in order to provide the locking mechanism described earlier.

In one form of the technology an electrical interface is formed between the electrical terminals on the air circuit 4170 and the electrical contacts 8004 on the RPT device 4000. This electrical interface is used to communicate information regarding the air circuit 4170 or peripherals such as patient interfaces 3000 connected thereto to the RPT device 4000.

### 5.9.1 Detection Circuits

In examples of the technology, the air circuit 4170 comprises a detection circuit configured to relay information to the RPT device. In certain forms, the information may relate to the air circuit 4170, a connected peripheral such as a patient interface 3000, and/or a connection state or a connection quality between the air circuit 4170. For example, the detection circuit may be configured to communicate the information using one or more of the wires in the air circuit 4170, or alternatively using one or more wireless technologies including but not limited to Bluetooth™, WiFi, RFID, or NFC.

FIGS. 9A and 9B show a first end 7004 of an air circuit 4170 in accordance with the present technology, which in this form is the end of the air circuit 4170 which is configured to connect to a peripheral device such as a patient interface 3000 in use. In the illustrated example, the air circuit 4170 comprises a fixture 9002 which receives or otherwise contains a detection circuit 9008. The fixture 9002 is shown as extending from an inner wall 9004 of the first cuff 7006, radially inwardly toward a longitudinal axis of the air circuit 4170. However, the foregoing should not be seen as limiting on the technology. For example, the detection circuit 9008 may be housed within the wall 9006 of the second cuff, or extend inwardly or outwardly of the first cuff 7006.

FIG. 9B shows a cross sectioned view of the first end 7004 of the air circuit 4170 of FIG. 9A. In this example the detection circuit 9008 can be seen within the fixture 9002. In some examples described herein this detection circuit 9008 comprises a printed circuit board containing electronic components 9010 which are configured to detect one or more parameters of a connected peripheral device as described herein.

The first cuff 7006 may include electrical connectors 9012 which provide a connection between the detection circuit 9008 and one or more wires within the conduit 7002.

In use the first cuff 7006 may be engaged with a connection port 3600 on a patient interface 3000. The connection port 3600 may engage with the inner wall 9004 of the first cuff 7006 to provide an airtight connection. Once the first cuff 7006 has been fully engaged with the connection port 3600, a stop surface 9014 may prevent the connection port 3600 from being inserted further.

Other features of the first cuff 7006 in certain forms of the technology may include a curved entry 9016 to facilitate an easier connection to a peripheral device, such as allowing less precise alignment between the first cuff 7006 and the elbow on a patient interface 3000, and a sealing retention bead 9018 or narrower region of the inner wall 9004 which may engage with the connection port 3600 to provide the airtight seal. It should be appreciated that any one or more of the features of the first cuff 7006 may also be used on the second cuff 7010 in certain forms to facilitate an airtight connection between the second cuff 7010 and the tubular connector 8002 on the RPT device 3000.

### 5.9.1.1 Passive Detection

In examples of the technology, the detection circuit comprises electrical conductors which, when electrically connected, either by an electrical short, a resistive component, or any other electronic components allow for the detection of one or more parameters of the connected peripheral device.

Examples of detection configurations 10000 which facilitate one or more of peripheral detection, and/or connection to a peripheral are shown in FIG. 10A-10K. In each of these examples, the detection circuit 9008 comprises two or more conductors 10002A, 10002B 10002C, which have exposed electrical terminals 10004A, 10004B, 10004C that are configured to contact one or more electrical terminals 10006A-10006F in the peripheral device 3000 (or component thereof such as the connection port 3600). In each of the examples 10A-10H the electrical terminals 10004A, 10004B, 10004C, extend inwardly of the inner wall 9004 of the first cuff 7006, while the electrical terminals 10006A, 10006B, 10006C extend outwardly of the peripheral device, such as from the connection port 3600 of a patient interface 3000.

In use the first cuff 7006 may be engaged with the connection port 3600, until the end 10008 of the connection port 3600 engages with the stop surface 9014. When engaged, the electrical terminals 10004A, 10004B, 10004C in the first cuff 7006 may contact the corresponding electrical terminals 10006A, 10006B, 10006C in the connection port 3600 to complete an electrical circuit.

FIG. 10A shows a form of the technology in which electrical conductors in the air circuit 4170 are bridged by a conductor 10010 in the peripheral device or patient interface 3000. The conductor 10010 in the peripheral device or patient interface 3000 completes the electrical circuit between the electrical terminals 10004A, 10004B when the air circuit 4170 is correctly engaged to the peripheral device or patient interface 3000 therefore enabling detection that a correct connection has been made.

In the form of FIG. 10B, the peripheral device comprises a resistive element 10012 which provides a resistance between the conductors in the peripheral device. When the peripheral device is connected to the air circuit 4170, this resistive element 10012 may be electrically connected between the electrical terminals 10004A. 10004B to facilitate detection of the connection state, for example by detecting a current flow or resistance value. In addition, by using resistive elements 10012 having different resistance values in different peripheral devices, it may be possible to measure the resistance value to detect which peripheral device (e.g. patient interface 3000) is connected. Techniques for measuring resistance are known to those skilled in the art such as by measuring current at a given supply voltage or connecting the resistive element 10012 as part of a voltage divider and measuring the resulting voltage.

Similarly, techniques for measuring impedance, including inductance and capacitance should be known to those skilled in the art and may be applied in certain forms. For example, the forms illustrated in FIGS. 10C and 10D operate in a similar manner to FIG. 10B, with the resistive element 10012 being replaced by the use of a capacitive component 10014 or inductive component 10016 respectively. For example, the capacitive component 10014 may be provided as a capacitor, for example two conductive elements in the peripheral device which have an insulating material between. The inductive component 10016 may be provided by an inductor, for example a coil of wire with a known inductance. The capacitive component 10014 and inductive component 10016 may be electrically connected to the electrical terminals 10006A, 10006B using any suitable method including wires or a printed circuit board (PCB).

In use, the capacitance or inductance values can be determined using methods known to those skilled in the art including by measuring the impedance provided to one or more AC waveforms. Accordingly, these examples allow for both peripheral connection detection, as well as identification by using different capacitive and inductive components in different peripheral devices.

FIG. 10E shows an example where three or more conductors 10006A, 10006B, 10006C in the peripheral device (e.g. patient interface 3000) are used as part of a detection circuit 9008 for determining connection state, as well as peripheral identification. In the example shown the electrical terminals 10004A, 10004B, 10004C in the air circuit 4170 connect to a voltage divider formed by a plurality of resistive elements 10012 in the peripheral device. Accordingly, by use of different resistive elements 10012 having different values of resistance, different voltages may be produced by the voltage divider. These different voltages may be configured to correspond to different peripheral devices (such as patient interfaces 3000) thereby facilitating detection and identification of the peripheral device connected.

It should be appreciated that, while the foregoing examples are shown with two or three conductors, this should not be seen as limiting on the technology, and in some examples more conductors may be used, while in other examples the conductors shown may be shared with conductors used for other purposes such as for heating the air circuit 4170. For example, with reference to FIG. 10E, the first conductor 10002A may be a heated conduit wire, which supplies a voltage to the voltage divider. The second conductor 10002B may be a comparatively low impedance return wire, while the third conductor 10002C may be a signal wire for receiving and relaying the divided voltage to the RPT device 4000.

FIGS. 10F and 10G, show forms of the technology comprising RC and LC resonant circuits respectively, wherein the detection and identification of the peripheral component may be achieved by determining the resonant frequency using methods known to those in the art. For example the peripheral device (such as a patient interface 3000) may comprise a resonant circuit tuned to a specific resonant frequency to facilitate detection, and/or identification of the peripheral device by a detection circuit 9008. In some examples the resonant circuit may comprise one or more inductors connected electrically in parallel with one or more capacitors. In other examples the resonant circuit may comprise one or more capacitors connected electrically in series with one or more resistors.

While the foregoing examples are each shown with discrete terminals in the peripheral device and air circuit 4170, this should not be seen as limiting on the technology. For example, the conductors may be provided around the circumference of the first cuff 7006 or peripheral device to allow peripheral device detection in any orientation. FIGS. 10H and 10I show examples of an end view of a connection port 3600 of a patient interface 3000 according to two forms of the technology, in which the electrical terminals 10006A, 10006B, 10006C are provided around the perimeter of the connection port 3600.

FIG. 10H shows a first electrical terminal 10006A which extends around less than half of the perimeter (for example between 40 and 49% of the perimeter) of the connection port 3600 (or elbow of a patient interface), and a second electrical terminal 10006B which extends substantially the same distance around the perimeter on the directly opposing half of the connection port 3600. In other words the terminals 10006A, 10006B are equally spaced around the perimeter of the connection port 3600. These electrical terminals 10006A, 10006B are electrically isolated from one another by small insulating gaps 10007 around the perimeter of the connection port 3600. The insulating gaps 10007 may be smaller than the size of the corresponding terminals 10004A, 10004B on the air circuit 4170, so as to ensure that the terminals do not electrically bridge or connect the electrical terminals 10006A, 10006B in use.

FIG. 10I shows an example comprising three electrical terminals 10006A, 10006B. 10006C which extend around less than a third of the perimeter (for example between 25 and 32% of the perimeter). The terminals are equally spaced, and electrically isolated by insulating gaps in a similar manner to FIG. 10H.

FIG. 10J shows one example wherein the peripheral device or patient interface 3000 comprises a plurality of electrical terminals 10006A, 10006B, 10006C, 10006D arranged along the length of the connection port 3600 of first cuff 7006 and able to be contacted at various positions along the length of the connection port or first cuff 7006. For example, by arranging a plurality of any one or more of a conductor, resistive, capacitive, or inductive elements between each of the terminals, it may be possible to detect how far well or how deeply the first cuff 7006 has been engaged with the connection port 3600. It should be appreciated that, while the plurality of terminals has been shown on the peripheral device or patient interface 3000, it may instead be advantageous to position the plurality of terminals along the first cuff 7006, as this can allow for production of the peripheral device or patient interface 3000 more cheaply.

In a further example of the technology shown in FIG. 10K, the electrical terminals 10006A, 10006B on the peripheral device or patient interface 3000 may have an elongated profile which extends longitudinally along the connection port 3600 or part thereof. For example the electrical terminals 10006A, 10006B may be configured to extend along at least 50% of the length of the connection port 3600. Similarly, the electrical terminals 10004A, 10004B on the first cuff 7006, may have an elongated provide which extends along the first cuff 7006, or part thereof such as along an inner wall 9004 of the first cuff 7006. For example the electrical terminals 10004A, 10004B may be configured to extend from the curved entry 9016 to the stop surface 9014 of the first cuff or part way thereof, such as at least 50% of the distance thereof.

While the foregoing examples are performed using discrete electronic components and terminals this should not be seen as limiting on the technology. For example, with reference to FIG. 10K, the resistive components shown may be provided by a strip of resistive material, such as carbon or graphite. In this example, the distance of insertion between the first cuff 7006 and connection port 3600 may determine the amount of resistive material (and therefore resistance) between the conductors of the air circuit 4170, and therefore use of a single resistive material/component can provide insertion information.

### 5.9.1.2 Magnetic Detection

Another feature of the present technology is providing a means to determine peripheral detection using magnetic fields.

In one example of the technology shown in FIG. 11A, the peripheral device (such as a patient interface 3000) is provided with a magnetic component 11002 or plurality of magnetic components. For example, the magnetic component 11002 is a component which generates a magnetic field. This may include a magnetic coating, a toroidal magnet, discrete magnets, electromagnets, or any combination thereof. The magnetic component 11002 may be positioned within any part of the peripheral device or patient interface 3000 such as the connection port 3600.

A corresponding detection circuit 9008 may be provided with one or more electronic components 9010 configured to respond to the magnetic field, for example these magnetic field devices can include a Hall-effect sensor, magnetometer, or reed switch. The detection circuit 9008 may be positioned or otherwise located such that, in use when the peripheral device is connected or disconnected from the air circuit 4170, the corresponding connection or disconnection can be detected in the detection circuit 9008.

For example, the detection circuit 9008 may be positioned within an inner wall 9006 of the first cuff, extending inwardly or outwardly of the inner wall 9006, or within a fixture 9008 as described herein. Similarly the magnetic component 11002 should be positioned in the peripheral device in such a way that the field generated by the magnetic component can be received and detected by the detection circuit 9008 in use.

In one example, the magnetic component 11002 is provided as part of the elbow on a patient interface 3000, and the detection circuit 9008 is provided as part of the first cuff 7008 of an air circuit 4170. Accordingly, when the elbow is connected to the first cuff 7008, the detection circuit is able to detect the presence of the magnetic field, and relay the information to the RPT device 4000 accordingly.

In another example, the magnetic component 11002 is releasably attachable to the peripheral device or patient interface 3000, and may be configured to attach to the peripheral device using adhesives, clips or any other suitable retention mechanism. For example the magnetic component may be releasably attached to the decoupling structure, elbow, plenum chamber, frame or any other component of the patient interface.

These magnetic technologies may be used to detect a connection or disconnection of an air circuit 4170 to a peripheral device, i.e. by determining the presence or absence of the magnetic field. In addition, in some forms of the technology, by varying the positioning, polarity and strength of the magnetic components used between different peripheral devices, the different peripheral devices may be detected and identified.

In a yet further example of the technology, the strength of the magnetic field detection may be used as an indication of connection quality or depth of insertion. For example, the distance between the magnetic component and the magnetic field detection device may be reduced as the coupling between the air circuit 4170 to the peripheral device improves.

For example, a Hall-effect sensor may be used to measure the magnetic field strength, and relay this information as a digitised value, and/or as a voltage, to the RPT device 4000. If the measured magnetic field strength is below a first predetermined threshold, the RPT device 4000 may be configured to perform an action associated with a disconnected peripheral device such or patient interface 3000, such as stopping, or not starting, the flow of breathable gas. If the measured magnetic field strength is above a second predetermined threshold the RPT device 4000 may be configured to perform an action associated with a properly connected peripheral device or patient interface 3000, such as initiating, resuming, or continuing delivery of the flow of breathable gas. If the measured magnetic field strength is between the first predetermined threshold and the second predetermined threshold, the RPT device 4000 may be configured to perform an action associated with a partially connected peripheral device or patient interface 3000, such as notifying the patient.

In other examples, the detection circuit 9008 may be provided with one or more reed switches configured to switch between open and closed states in the presence of a magnetic field of a predetermined magnetic field strength. In this way, the connection status of the peripheral device or patient interface 3000 may be communicated to the RPT device 4000 and actions performed accordingly. In some examples one or more reed switches may be provided to detect a lower intensity field strength which is indicative of a connected peripheral device, which has not been fully connected. For example, a first reed switch may be provided with a first magnetic field strength threshold indicative of a poorly connected peripheral device, and a second reed switch may be provided with a second magnetic field strength threshold indicative of a properly connected peripheral device. Accordingly, all three states may be communicated to the RPT device 4000. For example, when both reed switches are open, an open circuit may be provided to the RPT device 4000 indicating that no peripheral device is connected. When a peripheral device is partially connected, the first reed switch may close, connecting an electronic component between the wires 10002A, 10002B in the in the air circuit 4170, such as a resistor capacitor or inductor. This component may be detected by the RPT device and an action performed accordingly. When the peripheral device is properly connected to the air circuit 4170, the second reed switch may close providing a second electronic component or short in parallel with the first electronic component, thereby causing a change in the impedance which is detectable at the RPT device 4000 to allow the RPT device 4000 to perform an action accordingly.

In each of the foregoing examples, it should be understood that the thresholds may be provided or adjusted by changing either the relative positioning of the magnetic component 11002, and or detection circuit components. In other examples, the detection circuit components may be provided with different thresholds, or adjustable thresholds which may be tuned to specific peripheral device/air circuit 4170 configurations. In other examples, the detection circuit may be configurable to only communicate programmed threshold changes to the RPT device 4000. In further examples, the RPT device 4000 comprises adjustable thresholds which can be adjusted as required.

The polarity of the magnetic component 11002, can be detected using methods known to those in the art, such as using a Hall-effect sensor, magnetometer or reed switches positioned in different axes within the detection circuit 9008. Accordingly, in one form of the technology a certain type of peripheral device (such as nasal masks) may be provided with magnetic components 11002 having a first polarity, and a second type of peripheral device (such as full-face masks) may be provided with magnetic components 11002 having a second polarity, and the detection circuits 9008 described herein may be configured to relay the peripheral type information to the RPT device 4000 so that the RPT device 4000, may perform an action accordingly (such as altering the flow rate of the flow generator).

In a further example of the technology, Hall-effect latches may be used. For example, the peripheral device may comprise one or more magnetic components 11002 providing a first polarity magnetic field and a second polarity magnetic field. In use these magnetic fields may be configured such that, during connection of the air circuit 4170 to the peripheral device, the first magnetic field is dominant at the detection circuit 9008. For example this can be achieved by the relative positioning of the magnetic components 11002 to the Hall-effect latch. This signal can be detected using any of the means described herein, however in one example of the technology this field is configured to switch a Hall-effect latch into an open state. Once connected the second polarity magnetic field is configured to be dominant at the detection circuit 9008 resulting in the Hall-effect latch into a closed state. This Hall-effect latch remains in the closed state until removal or partial removal of the peripheral device causes the first magnetic field to become dominant at the detection circuit 9008, this causes the Hall-effect latch to revert to the opened state. Accordingly, the RPT device 4000 is able to determine when the peripheral device is correctly connected, or has been disconnected or partially disconnected.

In each of the foregoing examples, the information detected by the detection circuit 9008 can be related to the RPT device 4000, using any suitable methods known to those in the art, including digital communications such as serial, I²C, SPI, CAN, Ethernet, Wifi, Bluetooth, etc. Or using analogue signals such as voltages, for example the magnetic field strength may be communicated as an absolute voltage ranging between 0 and 5V while the polarity of the voltage may be used to communicate magnetic component 11002 polarity.

It should be appreciated that reference herein to magnetic components includes permanent magnets, as temporary magnetic materials and electromagnets. For example, in one embodiment of the technology, power may be provided to the peripheral device or patient interface 3000 via one or more wires in the air circuit 4170. The power may be used to power an electromagnet, and the resulting field strength detected by a detection circuit as described herein.

One advantage of using magnetic technologies is that the magnetic component 11002 may not need to make contact with the magnetic detection circuit 9008, and therefore the magnetic components and detection circuits described herein can be positioned in any suitable locations including but not limited to: within the walls of the first cuff 7006 or peripheral device such as a patient interface 3000, for example by over moulding or a similar process; on an internal or external wall of the cuff 7006 or patient interface 3000, for example by using a fastener or adhesive or in a fixture 9002 as described herein.

FIG. 11B shows an example of the technology wherein the air circuit 4170 comprises conductors 10002A, 10002B which have a ferromagnetic component, comprise a ferrous material, or otherwise comprise a component with a relative magnetic permeability greater than 1. For example, a steel strip may be included in the air circuit 4170, or in the case of a heated conduit, one or more of the wires in the heated wire circuit may be constructed of an iron containing alloy such as an aluminium-iron alloy. These ferrous conductors may be used to couple the magnetic field from the magnetic device to a detection circuit of the RPT device 4000. Accordingly, this may allow for cheaper air circuits 4170 which facilitate peripheral detection without dedicated detection circuits as described herein.

It may be advantageous for the conductors 10002A, 10002B, to be positioned on two or more sides of the magnetic component 11002 to thereby facilitate conduction of both magnetic polarities to the detection circuit. For example, the conductors 10002A, 10002B may be positioned such that, when the peripheral device and first cuff 7006 are connected, each conductor is on an opposing side of the magnetic component 11002. In another example, a first conductor 10002A, may be configured to lie substantially in a first axis, and a second conductor 10002B may be configured to lie substantially in a second axis, the first axis being approximately at 90 degrees to the second axis. The magnetic component may be provided at a 45 degree angle relative to both the first axis and the second axis, such that at least part of the corresponding positive and negative magnetic fields may be magnetically coupled through the conductors 10002A, 10002B.

In a yet further example of the technology shown in FIG. 11C, an air circuit 4170 is provided with both a detection circuit 9008 and one or more magnetic components 11002. In use, insertion of part of a peripheral device such as a patient interface 3000 into the first cuff 7006, results in the position of one or more of the magnetic components 11002 changing relative to the detection circuit 9008. For example, the magnetic component 11002 may be a toroidal magnet, or otherwise comprise a ring-shaped component. This magnetic component 11002 may be movably coupled to an inner wall 9004 of the first cuff 7006, such that it can move longitudinally along the length of the first cuff 7006. The magnetic component 11002, may be biased towards the end of the first end 7004, using at least one basing element 11004 or resiliently deformable component, such as a spring.

In use, insertion of the peripheral device such as a patient interface 3000 into the first cuff 7006 may result in the end 10008 of the peripheral device engaging magnetic component 11002 or moveable component carrying the magnetic component, urging the magnetic component 1102 inwardly towards the detection circuit 9008. This movement can be detected as a change in magnetic field strength, and therefore can be used to determine the positioning of the peripheral device relative to the first cuff 7006.

The foregoing allows for the present technology to be used to detect insertion depth of peripheral devices such as patient interfaces 3000, without requiring any modifications to the peripheral devices, and can therefore be backwards compatible with existing peripheral devices.

### 5.9.1.3 Radio Frequency Detection

In a yet further example of the technology shown in FIG. 12, the peripheral device such as a patient interface 3000 may comprise a radio frequency (RF) transponder or tag 12002. The RF tag 12002 may be programmed to store information about the peripheral device such as the model, or manufacture date. This information may be read using a RF transceiver as part of a detection circuit 9008 as described herein.

In one example of the technology the RF transceiver is provided by the detection circuit 9008. That is to say that the detection circuit comprises an RF antenna, drive circuit and electronics configured to read and/or write information to and from the RF tag 12002 The RF transceiver may be powered by one or more electrical conductors in the air circuit 4170, and may be configured to transmit the peripheral device information to the RPT device 4000, by using any of the wired or wireless methods described herein.

In an alternative form of the technology, the detection circuit 9008 may be configured to comprise an RF antenna circuit, and be operatively connected to one or more other RF components within the RPT device 4000 using one or more wires within the air circuit 4170. For example the RPT device 4000 may comprise the drive circuit and electronics configured to read and/or write information to and from the RF tag 12002.

One advantage of the use of radio frequency technologies as described herein, may be that the RF tag may be updated with additional information during use. For example, the number of cycles, hours or days the peripheral device such as a patient interface 3000 has been used for. This may be used to generate reminders as to when the peripheral device should be replaced.

It should be appreciated that the RF tag 12002 does not need to be positioned immediately adjacent to the detection circuit 9008, as the detection circuit may be configured to provide an RF field capable of reading the information on the tag wirelessly. Accordingly, use of RF technologies may advantageously allow for less precise positioning of the peripheral device relative to the first cuff 7006 while still allowing for detection as to whether the peripheral device and first cuff 7006 are connected.

### 5.9.1.4 Backwards Compatibility

In examples of the technology where a heated air circuit 4170 is used, it may be advantageous for the heated air circuit 4170 to continue to operate when connected to peripheral devices which do not have certain of the components or features described herein that may be used for detecting the peripheral component. In other words, the air circuit 4170 may comprise technologies which enable detection of the peripheral devices using mechanical means. One example of this is shown in FIG. 11C.

A further example of mechanical detection is shown in FIG. 13, wherein the detection circuit 9008 comprises a switch 13002. In use the switch 13002 engages with a feature on the peripheral device such as a patient interface 3000 such as the end of the connection port 3600 to complete an electrical circuit. The switch 13002 may be biased to an open or closed position such that, on removal of the patient interface 3000 the switch changes state automatically which can be detected by the detection circuit 9008.

In other examples of the technology the peripheral device such as a patient interface 3000 may be provided with a releasable magnetic component 11002 which may be attached to facilitate the magnetic detection technologies described herein. For example, a magnetic component 11002 may be clipped or otherwise fastened to the peripheral device in order to facilitate magnetic detection as described herein.

Another application of the foregoing technology is providing a system which allows for automatic configuration of RPT device 4000 settings based on detecting a known peripheral. For example, flow rates, humidity settings etc may be automatically set for a given peripheral device or patient interface 3000. These may be further customised on a user-by-user basis. In the event that a peripheral device is unable to be detected by the detection circuit the present technology may prompt a user to manually identify the peripheral connected, or configure the flow settings themselves. In some applications there may be a default profile for peripheral devices which are unable to be detected, such that manual configuration is not required.

### 5.9.1.5 Detection Examples

While the foregoing disclosure is primarily in relation to detecting peripheral components which are generally connected to the end of an air circuit 4170, this should not be seen as limiting on the technology. For example, the components of the present technology, such as the detection circuits described herein may instead be located with the RPT device 4000, and configured to detect the connection of any components to the RPT device 4000, such as air circuits 4170, humidifies, supplementary oxygen or gas sources etc.

Similarly, while the present disclosure discusses detecting a patient interface 3000 at the end of an air circuit 4170, the technology may similarly be applied to detect other peripheral components such as short tubes, which may be connected to the end of the air circuit 4170.

Furthermore, the present technology may be used to detect, and in some cases identify, a plurality of peripheral devices, for example the technologies described herein can be used to both identify the type of air circuit 4170 used, as well as the peripheral device, and provide an indication of the relative coupling between same.

It should also be understood that, while the examples described herein are described in relation to a first cuff 7006 being connected to a patient interface 3000, in some examples of the technology the technology may be used to detect removal of any component of a patient interface 3000. For example, FIG. 14 shows a patient interface 3000 with a removable connection port 3600. The detection circuits 9008 described herein may be configured to detect any part of the patient interface 3000, for example by positioning a magnetic component 11002 or RF tag 12002 on the patient interface 3000. In other examples, the technologies described herein, such as using switches, or passive detection technologies may be used to detect the connection between the connection port 3600 and patient interface 3000. In other words the connection port 3600 may be electrically connected to the air circuit 4170 in use so as to facilitate communication of the connection state between the connection port 3600 and patient interface 3000 to the RPT device 4000 in use.

### 5.9.2 Actions

On detection of any one or more of: a peripheral device being connected or disconnected; a certain type of device being identified; a change in connection quality, or a connection quality which is above or below a predetermined threshold, the RPT device 4000 may be configured to perform one or more actions. In certain forms of the technology the one or more actions may comprise any one or more of the following actions:
Generate an alert such as a visual, or audible alert.
Generate a message such as a visual message output on a display, or audible message, or send a message to an external device.
Change one or more operating parameters of the RPT device, for example changing the flow rate, any one or more parameters relating to the heating of the air circuit 4170 or humidification of air.
Stopping or starting the operation of the RPT device 4000.
Logging or otherwise recording the information.

One advantage of certain forms of this technology is, for example, that a patient may disconnect the air circuit 4170 from the peripheral device or patient interface 3000 in the middle of the night to use the bathroom (whether this is by disconnecting the air circuit 4170 from the connection port 3600/elbow or the connection port 3600/elbow from the patient interface). This disconnection may be detected using the technologies described herein, and the RPT device 4000 may be stopped until connection is detected once more. Similarly, when the air circuit is connected, the RPT device may be automatically started.

In a further example of the technology, the detection circuit 9008 may provide feedback to the RPT device 4000 that there is a poor connection between the air circuit 4170 and the peripheral device or patient interface 3000. The RPT device 4000 can then determine the appropriate action to perform. For example, if the RPT device 4000 may provide an audio or visual alert. If the RPT device 4000 detects that the patient is asleep, or the time of day is one in which the patient is likely to be asleep, the RPT device 4000 may generate a silent alert, and/or log the information and/or send the information to an external device in order to provide feedback once the patient is awake.

In a further example of the technology, the RPT device 4000 may be configured to avoid waking the patient during their deep sleep or REM sleep, and wake the patient during a light part of their sleep cycle. For example, to remedy a connection issue.

Another feature of the present technology is the ability to log peripheral connection data, to correlate air leakage information due to improper peripheral connection. Accordingly, the present technology may allow for peripheral devices, air circuits, and/or user instructions to be improved over time.

Another feature of the present technology is to feed peripheral connection quality information to the algorithms used to control the flow generator. Accordingly, flow rate adjustments may be made to account for variations in peripheral/air circuit connection quality.

### 5.10 Glossary

For the purposes of the present technology disclosure, in certain forms of the present technology, one or more of the following definitions may apply. In other forms of the present technology, alternative definitions may apply.

### 5.10.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H2O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH2O, g-f/cm2 and hectopascal. 1 cmH2O is equal to 1 g-f/cm2 and is approximately 0.98 hectopascal (1 hectopascal=100 Pa=100 N/m2=1 millibar˜0.001 atm). In this specification, unless otherwise stated, pressure is given in units of cmH2O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 5.10.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240

Polycarbonate: a thermoplastic polymer of Bisphenol-A Carbonate.

### 5.10.2 Patient Interface

Anti-asphyxia valve (AAV): The component or sub-assembly of a mask system that, by opening to atmosphere in a failsafe manner, reduces the risk of excessive CO2 rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Headgear: Headgear will be taken to mean a form of positioning and stabilizing structure designed for use on a head. For example the headgear may comprise a collection of one or more struts, ties and stiffeners configured to locate and retain a patient interface in position on a patient's face for delivery of respiratory therapy. Some ties are formed of a soft, flexible, elastic material such as a laminated composite of foam and fabric.

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 5.11 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

5.12 REFERENCE SIGNS LIST

| | |
|---|---|
| patient | 1000 |
| bed partner | 1100 |
| patient interface | 3000 |
| seal forming structure | 3100 |
| plenum chamber | 3200 |
| chord | 3210 |
| superior point | 3220 |
| inferior point | 3230 |
| positioning and stabilising structure | 3300 |
| vent | 3400 |
| connection port | 3600 |
| forehead support | 3700 |
| patient interface | 3800 |
| RPT device | 4000 |
| external housing | 4010 |
| upper portion | 4012 |
| lower portion | 4014 |
| panel | 4015 |
| chassis | 4016 |
| handle | 4018 |
| pneumatic block | 4020 |
| air filter | 4110 |
| inlet air filter | 4112 |
| muffler | 4120 |
| outlet muffler | 4124 |
| pressure generator | 4140 |
| blower | 4142 |
| motor | 4144 |
| anti-spillback valve | 4160 |
| air circuit | 4170 |
| supplementary gas | 4180 |
| electrical components | 4200 |
| PCBA | 4202 |
| electrical power supply | 4210 |
| input device | 4220 |
| transducers | 4270 |
| humidifier | 5000 |
| humidifier inlet | 5002 |
| humidifier outlet | 5004 |
| humidifier base | 5006 |
| humidifier reservoir | 5110 |
| conductive portion | 5120 |
| reservoir dock | 5130 |
| locking lever | 5135 |
| water level indicator | 5150 |
| heating element | 5240 |
| conduit | 7002 |
| First end | 7004 |
| First cuff | 7006 |
| Second end | 7008 |
| Second cuff | 7010 |
| first locking half | 7012 |
| terminals | 7014 |
| ribs | 7016 |
| valleys | 7018 |
| grip portions | 7020 |
| identifying components | 7022 |
| markings | 7024 |
| tubular connector | 8002 |
| electrical contacts | 8004 |
| second locking half | 8006 |
| fixture | 9002 |
| inner wall | 9004 |
| wall | 9006 |
| detection circuit | 9008 |
| electronic components | 9010 |
| electrical connectors | 9012 |
| stop surface | 9014 |
| curved entry | 9016 |
| sealing retention bead | 9018 |
| Detection configurations | 10000 |
| conductors | 10002A-B |
| electrical terminals (air circuit) | 10004A-C |
| electrical terminals (peripheral device) | 10006A-D |
| Insulating gap | 10007 |
| end | 10008 |
| conductor | 10010 |
| resistive element | 10012 |
| capacitive component | 10014 |
| inductive component | 10016 |
| magnetic component | 11002 |
| basing element | 11004 |
| RF tag | 12002 |
| switch | 13002 |

1.-29. (canceled)

30. An air circuit for a respiratory therapy system, the air circuit comprising:
a conduit configured to receive a flow of breathable gas from a respiratory therapy device in use;
a first cuff provided to a first end of the conduit to facilitate connection to a peripheral device in use,
a second cuff provided to a second end of the conduit, to facilitate connection to the respiratory therapy device in use, and
a detection circuit,
wherein the detection circuit is configured to detect the presence or absence of the peripheral device in use, and to communicate information regarding the presence or absence of the peripheral device to the respiratory therapy device.

31. The air circuit of claim 30, wherein the first cuff is configured to connect to a patient interface.

32. The air circuit of claim 30, wherein the second cuff is configured to connect to a tubular connector on the respiratory therapy device.

33. The air circuit of claim 30, wherein the detection circuit comprises electrical conductors which when electrically connected provide a signal indicative of a connection to the peripheral device.

34. The air circuit of claim 33, wherein the electrical conductors are configured to electrically connect to one or more passive components in the peripheral device.

35. The air circuit of claim 30, wherein the first cuff comprises two or more electrical terminals, configured to connect to corresponding terminals in the peripheral device.

36. The air circuit of claim 30, wherein the second cuff comprises two or more electrical terminals, configured to connect to corresponding terminals in the respiratory therapy device.

37. The air circuit of claim 30, wherein the detection circuit is configured to detect a magnetic component in the peripheral device.

38. The air circuit of claim 30, wherein the detection circuit is provided in the first cuff.

39. The air circuit of claim 38, wherein the detection circuit comprises a switch configured to detect the insertion or removal of the peripheral device from the first cuff.

40. A respiratory therapy system comprising:
a respiratory therapy device configured to generate a flow of breathable gas;
an air circuit configured to receive the flow of breathable gas from the respiratory therapy device; and
a patient interface configured to facilitate delivery of the breathable gas from the air circuit to a patient's airways in use,
wherein the air circuit comprises a detection circuit which is configured to detect one or more features of the patient interface, and communicate information regarding the patient interface to the respiratory therapy device,
wherein the respiratory therapy device is configured to provide the flow of breathable gas when the information indicates that the patient interface is connected to the air circuit, and not provide the flow of breathable gas when the information indicates that the patient interface is disconnected from the air circuit.

41. The respiratory therapy system of claim 40, wherein the detection circuit comprises electrical conductors which when electrically connected provide a signal indicative of a connection of the air circuit to the patient interface.

42. The respiratory therapy system of claim 42, wherein the electrical conductors are configured to electrically connect to one or more passive components in the patient interface.

43. The respiratory therapy system of claim 40, wherein the detection circuit is configured to detect a magnetic component in the patient interface.

44. The respiratory therapy system of claim 40, wherein the detection circuit is provided in a first cuff of the air circuit.

45. The respiratory therapy system of claim 44, wherein the detection circuit comprises a switch configured to detect the connection or removal of the patient interface from the first cuff.

46. A respiratory therapy device comprising;
a flow generator configured to generate a flow of breathable gas; and
an outlet configured to communicate the flow of breathable gas to an air circuit in use,
wherein the flow generator comprises a detection circuit configured to detect the connection or disconnection of the air circuit from the outlet, and
wherein the flow generator is configured to provide the flow of breathable gas when the air circuit is connected, and not provide the flow of breathable gas when the air circuit is disconnected.

47. The respiratory therapy system of claim 46, wherein the detection circuit comprises electrical conductors which when electrically connected provide a signal indicative of a connection to the air circuit.

48. The respiratory therapy system of claim 47, wherein the electrical conductors are configured to electrically connect to one or more passive components in the air circuit.

49. The respiratory therapy system of claim 46, wherein the detection circuit is configured to detect a magnetic component in the air circuit.
